(19) United States
(12) Patent Application Publication
(10) Pub. No.: US 2024/0018070 A1
(43) Pub. Date: Jan. 18, 2024

(54) A METHOD FOR PRODUCING A CRACKING PRODUCT FRACTION COMPRISING PROPYLENE, C4 OLEFINS, OR BOTH

(71) Applicant: Neste Oyj, Espoo (FI)

(72) Inventors: Antti OJALA, Porvoo (FI); John JAMIESON, Porvoo (FI); Imane HACHEMI, Porvoo (FI); Marja TIITTA, Porvoo (FI); Eero KORHONEN, Porvoo (FI); Marja-Liisa KÄRKKÄINEN, Porvoo (FI); Eveliina MÄKELÄ, Porvoo (FI)

(73) Assignee: Neste Oyj, Espoo (FI)

(21) Appl. No.: 18/251,439

(22) PCT Filed: Oct. 29, 2021
(86) PCT No.: PCT/FI2021/050733

§ 371 Date: May 2, 2023

(30) Foreign Application Priority Data
Nov. 6, 2020 (FI) 20206117
Nov. 6, 2020 (FI) 20206120

(51) Int. Cl. C07C 4/06; C07C 1/24; C07C 7/00

(52) U.S. Cl. CPC C07C 4/06; C07C 1/24; C07C 7/00; C07C 2529/40

(57) ABSTRACT

The present disclosure provides a method for producing a cracking product fraction including propylene, C4 olefins, or both. In the method, a catalytic cracking feedstock containing a hydrocarbon feed including at least 5 wt-% isoparaffins is subjected to catalytic cracking in a catalytic cracking reactor.

### TECHNICAL FIELD

The present disclosure generally relates to catalytic cracking. The disclosure relates particularly, though not exclusively, to fixed bed or moving solid catalyst based catalytic cracking of a feedstock comprising isoparaffins to produce a cracking product fraction comprising propylene, C4 olefins, or both.

### BACKGROUND

This section illustrates useful background information without admission of any technique described herein representative of the state of the art.

Propylene and C4 olefins are important raw materials for the production of polymers and many organic chemicals. Propylene is one of the largest volume chemicals produced globally.

Typically, propylene and C4 olefins for the chemical industry originates from cracking of fossil feedstocks. Cracking is a refining process involving decomposition and recombination of organic compounds, especially hydrocarbons, affected by heat, pressure and optionally a catalyst. If the cracking is carried out by heat alone, it is called thermal cracking. If a catalyst is used, it is called catalytic cracking.

WO2009130392A1 discloses catalytic cracking of hydrogenated natural fats to gasoline range components.

Presently, an efficient process for producing renewable or bio-based propylene and C4 olefins, particularly propylene, is lacking.

### SUMMARY

It is an aim to solve or alleviate at least some of the problems related to prior art. An aim is to provide an environmentally sustainable production process of propylene composition, C4 olefin compositions, or both,

The appended claims define the scope of protection.

According to a first aspect there is provided a method for producing a cracking product fraction comprising propylene, C4 olefins, or both, the method comprising:
providing a catalytic cracking feedstock comprising:
   a hydrocarbon feed comprising, based on the total weight of the hydrocarbon feed, at least 5 wt-% isoparaffins, the sum of the wt-% amounts of isoparaffins and n-paraffins in the hydrocarbon feed being at least 80 wt-%;
subjecting the catalytic cracking feedstock to catalytic cracking in a catalytic cracking reactor at a temperature selected from a range from 300° C. to 450° C. in the presence of a solid catalyst to obtain a cracking product; and
separating from the cracking product at least a fraction comprising propylene, C4 olefins, or both.

It has surprisingly been found that catalytic cracking in a catalytic cracking reactor, under the above reaction conditions, of a hydrocarbon feed comprising at least 5 wt-% isoparaffins is beneficial for producing propylene composition, C4 olefin compositions or both as it e.g. provides a high conversion normalised yield of propylene and C4 olefins (sum of the conversion normalised yields of propylene and C4 olefins), particularly, a high conversion normalised yield of propylene.

In certain embodiments, the catalytic cracking is performed without feeding added molecular hydrogen (H₂) to the catalytic cracking reactor.

In certain embodiments, the catalytic cracking is performed without feeding added steam or added water (H₂O) to the catalytic cracking reactor.

In certain embodiments, the hydrocarbon feed comprises isoparaffins at least 8 wt-% or at least 10 wt-%, preferably at least 15 wt-%, further preferably at least 20 wt-%, more preferably at least 30 wt-%, even more preferably at least 40 wt-% based on the total weight of the hydrocarbon feed. In certain advantageous embodiments, the hydrocarbon feed comprises isoparaffins at least 50 wt-%, preferably at least 60 wt-%, further preferably at least 65 wt-%, more preferably at least 70 wt-%, even more preferably at least 80 wt-%, most preferably at least 90 wt-%, such as at least 92 wt-% or at least 95 wt-%, based on the total weight of the hydrocarbon feed.

In certain embodiments, the hydrocarbon feed comprises, based on the total weight of the hydrocarbon feed, at least 50 wt-%, preferably at least 60 wt-%, further preferably at least 70 wt-%, more preferably at least 80 wt-%, and even more preferably at least 90 wt-% hydrocarbons having a carbon number of at least C10.

In certain embodiments, the hydrocarbon feed comprises, based on the total weight of the hydrocarbon feed, at most 5 wt-%, preferably at most 3 wt-%, more preferably at most 2 wt-%, even more preferably at most 1 wt-% hydrocarbons having a carbon number of at least C22.

In certain embodiments, the sum of the wt-% amounts of isoparaffins and n-paraffins in the hydrocarbon feed is at least 85 wt-%, preferably at least 90 wt-%, more preferably at least 95 wt-%, and even more preferably at least 99 wt-% based on the total weight of the hydrocarbon feed.

In certain embodiments, the hydrocarbon feed is a renewable hydrocarbon feed having a biogenic carbon content of at least 90 wt-%, preferably at least 95 wt-%, more preferably at least 100 wt-% based on the total weight of carbon in the hydrocarbon feed.

In certain embodiments, the catalytic cracking is performed at a pressure selected from a range from 0.01 MPa to 5.0 MPa.

In certain embodiments, the catalytic cracking is performed at a temperature selected from a range from 350° C. to 450° C. In certain embodiments, the catalytic cracking is performed at a temperature selected from a range from 370° C. to 450° C., preferably from 400° C. to 450° C., or at a temperature selected from a range from 350° C. to 430° C., preferably from 350° C. to 400° C., more preferably from 360° C. to 400° C.

In certain embodiments, the weight hourly space velocity (WHSV, mass flow of the catalytic cracking feedstock/catalyst mass) of the catalytic cracking is selected from a range from 0.01 to 10, preferably from 0.1 to 5. The unit of the WHSV is g catalytic cracking feedstock/g catalyst per hour.

In certain embodiments, one or more of temperature, pressure and WHSV, preferably at least temperature and WHSV, are controlled so that the conversion of catalytic cracking feedstock in the catalytic cracking is within a range from 0.20 to 0.85, preferably from 0.20 to 0.80.

In certain embodiments, the solid catalyst comprises zeolite or zeolite type material having a micropore size within the range from 4 to 6 Å, and wherein the solid catalyst preferably has medium strong acidity or strong acidity. In certain embodiments, the zeolite or zeolite type material comprises ZSM-5, MCM-22, SAPO-34 and/or p zeolite, preferably ZSM-5, MCM-22 and/or SAPO-34. The solid catalyst may comprise a carrier, that may be catalytically active, such as silica-alumina or clay, or catalytically inactive, such as silica, and/or further additives such as a binder, or any other commonly known catalyst additive(s).

In certain embodiments, the catalytic cracking reactor is a fixed bed reactor or a moving solid catalyst reactor, such as a fluidized bed catalytic cracking reactor or a moving bed catalytic cracking reactor.

In certain embodiments, the hydrocarbon feed is obtained by a process comprising:
hydrotreating renewable oxygen containing hydrocarbons, the renewable oxygen containing hydrocarbons preferably comprising one or more of fatty acids, fatty acid esters, resin acids, resin acid esters, sterols, fatty alcohols, oxygenated terpenes, and other renewable organic acids, ketones, alcohols, and anhydrides, the hydrotreating comprising deoxygenation and isomerisation reactions to obtain a hydrotreatment product comprising isoparaffins, and
removing vapour phase from the hydrotreatment product to obtain a vapour depleted hydrotreatment product and optionally providing the vapour depleted hydrotreatment product as the hydrocarbon feed.

In certain embodiments the method comprises recovering from the vapour depleted hydrotreatment product as the hydrocarbon feed a fraction comprising, based on the total weight of the fraction, at least 50 wt-%, preferably at least 60 wt-%, further preferably at least 70 wt-%, more preferably at least 80 wt-%, and even more preferably at least 90 wt-% hydrocarbons having a carbon number of at least C10.

In certain embodiments, the catalytic cracking feedstock comprises a recycled fraction separated from the cracking product.

In certain embodiments, the method comprises separating from the cracking product a fraction of hydrocarbons having a carbon number of at least C5, preferably at least C10.

In certain embodiments, the method comprises recycling at least a portion of the fraction of hydrocarbons having a carbon number of at least C5, preferably at least C10, to the catalytic cracking feedstock. The recycled portion of the fraction of hydrocarbons having a carbon number of at least C5, preferably at least C10, may be combined or mixed with the catalytic cracking feedstock before entering the catalytic cracking reactor, or be fed to the catalytic cracking reactor wherein it forms a part of the catalytic cracking feedstock. In certain embodiments, at least a portion of the fraction of hydrocarbons having a carbon number of at least C5, preferably at least C10, is, before recycling to the catalytic cracking feedstock, subjected to (partial) hydrotreatment, such as hydrogenation, for example to remove diolefins and/or purification treatment for example to remove at least aromatics.

In certain embodiments, the method comprises separating from the cracking product a fraction of C5-C9 hydrocarbons.

In certain embodiments, the sum of the wt-% amounts of the hydrocarbon feed and the recycled fraction in the catalytic cracking feedstock is at least 90 wt-%, preferably at least 95 wt-%, more preferably at least 99 wt-% based on the total weight of the catalytic cracking feedstock.

In certain embodiments, the method comprises purifying the fraction comprising propylene, or the fraction comprising both propylene and C4 olefins, to obtain purified propylene composition.

In certain embodiments, the method comprises fractionating the fraction comprising C4 olefins, or both propylene and C4 olefins, to obtain one or more of 1-butene, trans-2-butene, cis-2-butene, butadiene, isobutene as fractionated composition(s).

Different non-binding example aspects and embodiments have been illustrated in the foregoing. The embodiments in the foregoing are used merely to explain selected aspects or steps that may be utilised in different implementations. Some embodiments may be presented only with reference to certain example aspects. It should be appreciated that corresponding embodiments may apply to other example aspects as well.

### BRIEF DESCRIPTION OF THE FIGURES

Some example embodiments will be described with reference to the accompanying figures, in which:

FIG. 1 shows a schematic drawing of a method according to an example embodiment;

FIG. 2 shows a schematic drawing of a method according to an example embodiment.

### DETAILED DESCRIPTION

In the following description, like reference signs denote like elements or steps.

All standards referred to herein are the latest revisions available, unless otherwise mentioned.

Conversion may refer to the molar ratio of the compounds split in catalytic cracking into compounds having a smaller carbon number (converted feedstock) to the catalytic cracking feedstock subjected to the catalytic cracking (fed feedstock) (moles of converted feedstock/moles of fed feedstock).

However, in the context of the present disclosure an assumption is made regarding the amount of substance of the converted feedstock instead of using the amount of substance of actually converted feedstock, as that would be cumbersome to determine. Therefore, it is assumed herein that the cracking effluent fraction (cracking product fraction) of hydrocarbons having a carbon number of at least C10 constitutes the unconverted feedstock. The amount of substance (number of moles) of converted feedstock is then obtained by deducting the amount of substance (number of moles) of hydrocarbons having a carbon number of at least C10 in the cracking effluent (cracking product) from the amount of substance (number of moles) of the fed feedstock. The conversion is then determined by dividing this amount of substance of converted feedstock with the amount of substance of the fed feedstock, and if desired, the quotient may be multiplied with 100% to express the conversion as mol-%.

Conversion normalised yield refers herein to a yield expressed as the amount of substance (number of moles) of a certain compound or certain compounds in a cracking product normalised by the amount of substance (number of moles) of the converted feedstock, i.e. number of moles of a certain compound or certain compounds in a cracking product divided by number of moles of converted feedstock. In the context of this disclosure, the amount of substance of the converted feedstock is determined as described above. The conversion normalised yield may be expressed as mole percentage, namely 100%×(number of moles of a certain compound or certain compounds in a cracking product/number of moles of converted feedstock).

It is generally known that alkane and paraffin are synonyms and can be used interchangeably. Isoparaffins (i-paraffins) are branched, open chain paraffins, and normal paraffins (n-paraffins) are unbranched linear paraffins. In the context of this disclosure, the term “paraffin” refers to n-paraffins and/or isoparaffins. Similarly, the term “paraffinic” refers herein to compositions comprising n-paraffins and/or isoparaffins.

In certain embodiments, the isoparaffins have one or more C1-C9, typically C1-C2, alkyl side chains. Preferably, the side chains are methyl side chains, and the isoparaffins are mono-, di-, tri- and/or tetramethyl substituted.

The present disclosure provides a method for producing a cracking product fraction comprising propylene, C4 olefins, or both, the method comprising: providing a catalytic cracking feedstock comprising: a hydrocarbon feed comprising, based on the total weight of the hydrocarbon feed, at least 5 wt-% isoparaffins, the sum of the wt-% amounts of isoparaffins and n-paraffins in the hydrocarbon feed being at least 80 wt-%; subjecting the feedstock to catalytic cracking in a catalytic cracking reactor at a temperature selected from a range from 300° C. to 450° C. in the presence of a solid catalyst to obtain a cracking product; and separating from the cracking product a fraction comprising propylene, C4 olefins, or both.

It has surprisingly been found that catalytic cracking in a catalytic cracking reactor, under the aforementioned reaction conditions, of a hydrocarbon feed comprising at least 5 wt-% isoparaffins may result in a high conversion normalised yield, such as at least 44 mol-%, of propylene and C4 olefins (sum of the conversion normalised yields of propylene and C4 olefins). Particularly, a high conversion normalised yield of propylene, such as at least 20 mol-%, may be obtained. Also, molar ratio of propylene to all C3 compounds may be high, such as at least 0.7. In other words, high quality refinery grade propylene compositions may be obtained directly by fractionating from the cracking product a fraction of C3 compounds without additional efforts to reduce the amount of propane in said fraction, although some purification may be beneficial also in these cases. Furthermore, additional purification steps to achieve, from the fraction of C3 compounds, propylene compositions of chemical grade purity containing about 90-95 wt-% propylene, or even polymer grade purity containing propylene about 99 wt-% or more, require less effort, less expensive equipment and less energy (compared to fractions having lower molar ratios of propylene to total C3). Also, the molar ratio of C4 olefins to all C4 compounds may be high, such as at least 0.8. C4 olefins are obtainable from these C4 fractions with additional purification steps requiring less expensive equipment and less energy (compared to fractions having lower molar ratios of C4 olefins to total C4). Because methane is a strong greenhouse gas, a further advantage of the method of the present disclosure is that the conversion normalised yield of methane (C1) may be very low, particularly compared to cracking products obtained by steam cracking. The conversion normalised yield of methane may be 1 mol-% or less. Also, the conversion normalised yield of C2 hydrocarbons may be low, such as less than 3 mol-%, and the molar ratio of propylene to ethylene may be high, even above 17. C2 compounds are often considered less valuable than C3 and C4 compounds, particularly propylene and C4 olefins, especially when considering cracking products having biogenic carbon content. Furthermore, due to the relatively low temperature during the catalytic cracking, a very low amount of or no propadiene is formed. This is beneficial as C3 dienes polymerise very easily forming deposits and/or causing fouling of the cracking catalyst. With the present method, more of valuable products may be obtainable from the feedstock, for example compared to steam cracking, which is the dominant technology for producing propylene from fossil feedstocks, steam cracking producing propylene only as a desired by-product of ethylene, along with significant amount of waste by-products such as CH₄. More of valuable products being obtainable from the feedstock is especially advantageous when the catalytic cracking feedstock comprises high value components, especially renewable hydrocarbon feed, so that merely small amounts of the high value components of the feedstock are wasted to lower value products. Particularly propylene is not trivial to manufacture from renewable or biogenic feedstocks.

The hydrocarbon feed comprises mainly, or preferably consists essentially of, hydrocarbons and comprise at most very small amounts or traces of heteroatoms. For example, the hydrocarbon feed may comprise at least 95 wt-%, preferably at least 98 wt-%, even more preferably at least 99 wt-% hydrocarbons based on the total weight of the hydrocarbon feed. In certain preferred embodiments, the hydrocarbon feed comprises at most 1 wt-% elemental oxygen, at most 60 wt-ppm elemental nitrogen, at most 10 wt-ppm, preferably at most 6 wt-ppm, more preferably at most 1 wt-ppm elemental sulphur, at most 50 wt-ppm, preferably at most 10 wt-ppm, more preferably at most 1 wt-ppm alkali and alkaline earth metals in total, based on the total weight of the hydrocarbon feed. Alkali and alkaline earth metals may neutralise acid sites of the solid catalysts. Also, alkali and alkaline earth metal ions increase coke formation in the catalytic cracking. The sulphur (S) content may be determined for example in accordance with ASTM-D5453, ASTM-D6667, or ENISO20846. The nitrogen (N) content may be determined for example in accordance with ASTM-D5762, or ASTMD4629. The oxygen (O) content may be determined for example in accordance with ASTM-D5622. The alkali and alkaline earth metal content may be determined for example in accordance with ASTMD8110-17. Without being bound to a theory, it is believed that more severe process conditions, such as higher temperature, would be needed, compared to those used in the present method, to cause efficient removal of covalently bound heteroatoms in the catalytic cracking feedstock. It is believed that in case the heteroatom amounts in the hydrocarbon feed would be much higher than the preferred amounts mentioned above, smaller heteroatom-containing moieties, such as shorter alcohols, thiols etc, would start to form through cracking of heteroatom-containing organic compounds and would end up in the cracking product. Depending on the desired end-use, and the specifications that the cracking product fractions would need to meet, cumbersome purification steps of the cracking product fractions could then be required.

Aromatics do not form, or form only very little, propylene and C4 olefins when subjected to catalytic cracking according to the method of the present disclosure. Also, aromatics and other impurities, such as heteroatoms, especially N, O and S, and unsaturated hydrocarbons, especially dienes, in the catalytic cracking feedstock increase the formation of coke during catalytic cracking. Coke formed during catalytic cracking tends to deposit on the solid catalyst causing deactivation of the catalyst. Typically, in fixed bed reactors, the solid catalyst is not regenerated during the catalytic cracking, but in separate regeneration cycles during which catalytic cracking cannot be performed in the fixed bed reactor being regenerated. When using moving solid catalyst reactors, it is possible to regenerate the solid catalyst during the catalytic cracking, e.g. in a separate coke burning reactor. However, repeated regeneration cycles may decrease the catalyst activity via various mechanisms, such as sintering, especially as the regeneration temperature is typically higher than the catalytic cracking temperature. Thus, a hydrocarbon feed or a catalytic cracking feedstock comprising only small amounts or preferably being essentially free from aromatics (and other impurities) are advantageous as the coke formation may be reduced and the yield of desired products, such as propylene and/or C4 olefins, increased. Accordingly, in certain preferred embodiments, the hydrocarbon feed comprises less than 1 wt-%, preferably less than 0.5 wt-% aromatics, and preferably less than 20 wt-%, more preferably less than 10 wt-%, even more preferably less than 1 wt-% naphthenes based on the total weight of the hydrocarbon feed. A low content of naphthenes in the hydrocarbon feed is beneficial as naphthenes in the feed decrease formation of propylene and C4 olefins in the catalytic cracking (compared to paraffins), and as naphthenes comprising a C6 ring are precursors for aromatics.

In certain embodiments, the sum of the wt-% amounts of isoparaffins and n-paraffins in the hydrocarbon feed is at least 85 wt-%, preferably at least 90 wt-%, more preferably at least 95 wt-%, and even more preferably at least 99 wt-% based on the total weight of the hydrocarbon feed. A highly paraffinic (isoparaffins and/or n-paraffins) hydrocarbon feed is preferred as it reduces coke formation and catalyst deactivation in the catalytic cracking and increases the yields of the desired products, such as propylene and C4 olefins. The remaining portion of the hydrocarbon feed, i.e. the portion that is not paraffins, may advantageously be hydrocarbons other than paraffins, for example olefins, naphthenes, and/or aromatics, and preferably contain at most very small amounts or traces of heteroatoms.

In certain embodiments, the hydrocarbon feed comprises isoparaffins at least 8 wt-% or at least 10 wt-%, preferably at least 15 wt-%, further preferably at least 20 wt-%, more preferably at least 30 wt-%, even more preferably at least 40 wt-% based on the total weight of the hydrocarbon feed. In certain advantageous embodiments, the hydrocarbon feed comprises isoparaffins at least 50 wt-%, preferably at least 60 wt-%, further preferably at least 65 wt-%, more preferably at least 70 wt-%, even more preferably at least 80 wt-%, most preferably at least 90 wt-%, such as at least 92 wt-% or at least 95 wt-% based on the total weight of the hydrocarbon feed. It has surprisingly been found that increasing the wt-% of isoparaffins in the hydrocarbon feed promotes the combined formation of the most desired products, namely propylene and C4 olefins, causing an increase in the sum of the conversion normalised yields of propylene and C4 olefins. Increasing the wt-% of isoparaffins in the hydrocarbon feed causes an increase particularly in the conversion normalised yield of propylene. For example, when the wt-% of isoparaffins in the hydrocarbon feed is at least 95 wt-%, the sum of the conversion normalised yields of propylene and C4 olefins may be 55 mol-% or more, and the conversion normalised yield of propylene may be 28 mol-% or more. Furthermore, it was found that increasing the wt-% of isoparaffins in the hydrocarbon feed may also decrease the formation of C5-C9 hydrocarbons usable e.g. as component for gasoline and/or solvent compositions, thinners, spot removers, etc., which may provide a convenient means for adjusting the conversion normalised yields of propylene and C4 olefins, and of the C5-C9 hydrocarbons, depending on their demand, price etc. by simply varying the wt-% of isoparaffins in the catalytic cracking feedstock.

A high isoparaffin content also provides the hydrocarbon feed with beneficial cold properties, such as a low cloud point. In certain embodiments, the cloud point of the hydrocarbon feed is 0° C. or lower, preferably −10° C. or lower, more preferably −15° C. or lower and even more preferably −25° C. or lower. A low cloud point reduces or may even omit the need to heat outdoor feed tanks, pipes and the like at low ambient temperatures.

In certain embodiments, the hydrocarbon feed comprises isoparaffins at most 98 wt-% or at most 95 wt-% based on the total weight of the hydrocarbon feed. Such hydrocarbon feeds are beneficial for catalytic cracking according to the method of the present disclosure.

In certain embodiments, the hydrocarbon feed comprises, based on the total weight of the hydrocarbon feed, at least 50 wt-%, preferably at least 60 wt-%, further preferably at least 70 wt-%, more preferably at least 80 wt-%, and even more preferably at least 90 wt-% hydrocarbons having a carbon number of at least C10. In certain particularly preferred embodiments, the hydrocarbon feed comprises, based on the total weight of the hydrocarbon feed, at least 93 wt-%, preferably at least 95 wt-%, more preferably at least 97 wt-% hydrocarbons having a carbon number of at least C10. Hydrocarbon feeds comprising mainly hydrocarbons having a carbon number of at least C10 are particularly suitable for the production of propylene and C4 olefins by catalytic cracking according to the method of the present disclosure. Longer saturated hydrocarbons crack at less severe conditions, compared to shorter saturated hydrocarbons. With this kind of hydrocarbon feed it is possible to produce a broad variety of different cracking product fractions with good conversion normalised yields. A hydrocarbon feed comprising mainly hydrocarbons having a carbon number of at least C10 yield a cracking product with, in addition to a high sum of the conversion normalised yields of propylene and C4 olefins, a high conversion normalised yield of C5-C9 hydrocarbons usable e.g. as component for gasoline and/or solvent compositions, thinners, spot removers, for metathesis reactions, as (co)monomers in polymers, or when manufacturing lube oil additives, surfactants, agricultural chemicals, coatings or corrosion inhibitors. Additionally, a cracking product fraction comprising cracked and unconverted hydrocarbons having a carbon number of at least C10 is obtained, which fraction may have increased isoparaffin content compared to the same carbon number fraction of the fresh hydrocarbon feed. Thus, the cracking product fraction comprising the unconverted hydrocarbons having a carbon number of at least C10 may be valuable as a recycle feed further enhancing the conversion normalised yields of propylene and C4 olefins. The unconverted fraction of hydrocarbons having a carbon number of at least C10 may have value added use e.g. by recycling it back to the catalytic cracking feedstock or as a component for aviation and/or diesel fuel compositions, optionally after a hydrotreatment such as hydrogenation of olefins. Such optional hydrotreatment may reduce coke-formation when the unconverted fraction of hydrocarbons is used for recycling, or allow higher shares of the fraction to be incorporated in aviation and/or diesel fuel compositions. As mentioned, for the purposes of calculating the conversion and the conversion normalised yield(s) it is assumed herein that the cracking effluent fraction (cracking product fraction) of hydrocarbons having a carbon number of at least C10 constitutes the unconverted feedstock.

In certain embodiments, the hydrocarbon feed comprises, based on the total weight of the hydrocarbon feed, at most 5 wt-%, preferably at most 3 wt-%, more preferably at most 2 wt-%, even more preferably at most 1 wt-% hydrocarbons having a carbon number of at least C22. Such hydrocarbon feeds are beneficial as C22 and larger hydrocarbons tend to increase coke formation in the catalytic cracking. Also, a more uniform feed, for example comprising mainly hydrocarbons in the range of carbon number C10-C21, allows adjustment or controlling of the catalytic cracking conditions so as to promote formation of propylene and/or C4 olefins.

In certain embodiments, the hydrocarbon feed comprises at least 70 wt-%, preferably at least 80 wt-%, more preferably at least 90 wt-% C14-C18 hydrocarbons based on the total weight of the hydrocarbon feed. Such hydrocarbon feeds are particularly advantageous for the production of propylene and C4 olefins by catalytic cracking according to the method of the present disclosure. Such very uniform feeds allow adjustment or controlling of the catalytic cracking conditions so as to promote formation of propylene and C4 olefins particularly well. With this kind of hydrocarbon feeds, it is possible to produce a broad variety of different cracking product fractions with good conversion normalised yields. For example, hydrocarbon feeds comprising a high amount of C14-C18 hydrocarbons yield a cracking product fraction of C5-C9 hydrocarbons usable e.g. as a component for gasoline and/or for chemical products intended for industry or households, such as in solvents, thinners and spot removers, in addition to the cracking product fraction(s) comprising shorter products including propylene and/or C4 olefins. Additionally, a fraction comprising cracked products mainly in the C10-C17 carbon number range and unconverted hydrocarbons mainly in the C14-C18 carbon number range (said fraction being for the purposes of calculating the conversion and conversion normalised yield assumed herein to be the unconverted feedstock) is obtained. Said fraction may have increased isoparaffin content compared to that of the fresh hydrocarbon feed. Accordingly, also this fraction comprising unconverted C14-C18 may have higher value as a recycle feed (recycled fraction) contributing to higher yield of propylene and C4 olefins in the catalytic cracking and/or as a component for aviation and/or diesel fuel compositions having good cold properties (compared to the fresh hydrocarbon feed), optionally after a hydrotreatment such as hydrogenation of olefins, for the same reasons as mentioned above for the unconverted fraction of hydrocarbons having a carbon number of at least C10.

In certain embodiments, the hydrocarbon feed has a boiling range within a range from 190° C. to 330° C. as determined according to ENISO3405. Such hydrocarbon feeds are particularly suitable for catalytic cracking according to the method of the present disclosure. Boiling range covers in the context of this disclosure a temperature interval from the initial boiling point, IBP, defined as the temperature at which the first drop of distillation product is obtained, to a final boiling point, FBP, when the highest-boiling compounds evaporate.

In certain embodiments, the hydrocarbon feed has a density within a range from 750 to 800 kg/m³, preferably from 775 to 795 kg/m³, at 15° C. as determined according to ENISO12185:1996. Such hydrocarbon feeds are particularly suitable for catalytic cracking according to the method of the present disclosure.

In addition to the hydrocarbon feed (fresh feed), the catalytic cracking feedstock may optionally comprise a recycled fraction separated from the cracking product. In other words, in certain embodiments, the catalytic cracking feedstock comprises a recycled fraction separated from the cracking product.

In certain embodiments, the catalytic cracking feedstock comprises, based on the total weight of the catalytic cracking feedstock, at least 90 wt-% hydrocarbon feed and optional recycled fraction, in total. Preferably, the catalytic cracking feedstock comprises, based on the total weight of the catalytic cracking feedstock, at least 95 wt-%, more preferably at least 99 wt-%, hydrocarbon feed and optional recycled fraction, in total. The catalytic cracking feedstock may in certain embodiments consist essentially of the hydrocarbon feed, or of the hydrocarbon feed and the optional recycled fraction. Catalytic cracking feedstocks comprising such high amount of the hydrocarbon feed, or the hydrocarbon feed and the optional recycled fraction, are particularly suitable for catalytic cracking according to the method of the present disclosure resulting in a high conversion normalised yield of propylene and C4 olefins, particularly propylene.

The optional recycled fraction may comprise unconverted hydrocarbons, i.e. hydrocarbons that were not split in the catalytic cracking into compounds having a smaller carbon number. Although the carbon number of unconverted hydrocarbons remain unchanged during catalytic cracking, even a significant portion may have reacted chemically. For example, unconverted hydrocarbons, particularly n-paraffins and/or naphthenes, may in the catalytic cracking react into isoparaffins. Accordingly, the recycled fraction may have a high isoparaffin content. In certain embodiments wherein the catalytic cracking feedstock comprises a recycled fraction, the wt-% amount of isoparaffins in the recycled fraction is at least the same as the wt-% amount of isoparaffins in the hydrocarbon feed, preferably the wt-% amount of isoparaffins in the recycled fraction is larger than the wt-% amount of isoparaffins in the hydrocarbon feed. The wt-% amount of isoparaffins in the recycled fraction is calculated based on the total weight of the recycled fraction and the wt-% amount of isoparaffins in the hydrocarbon feed is calculated based on the total weight of the hydrocarbon feed. In embodiments wherein the wt-% amount of isoparaffins in the recycled fraction is at least the same as that of the hydrocarbon feed, the recycled feed does not reduce, and advantageously even increases, the isoparaffin content of the catalytic cracking feedstock, which promotes the formation of propylene and C4 olefins, particularly of propylene, in the catalytic cracking. As mentioned, for the purposes of calculating the conversion and conversion normalised yield(s) it is assumed herein that the cracking effluent fraction (cracking product fraction) of hydrocarbons having a carbon number of at least C10 constitutes the unconverted feedstock.

The wt:wt ratio of the hydrocarbon feed (fresh feed) to the recycled fraction in the catalytic cracking feedstock may vary. For example, in certain embodiments the wt:wt ratio of the hydrocarbon feed to the recycled fraction in the catalytic cracking feedstock may be up to 1:10, such as up to 1:5 or up to 1:4. Preferably, the wt:wt ratio of the hydrocarbon feed to the recycled fraction in the catalytic cracking feedstock is within a range from 1:1 to 1:10. The overall yield of desired products may be increased by including in the catalytic cracking feedstock a significant portion of a recycled feed separated from the cracking product (recycled fraction). Also, because the isoparaffin content of the recycled fraction is often high, providing a catalytic cracking feedstock comprising more of the recycled fraction than of the hydrocarbon feed (fresh feed) is advantageous as it tends to increase the wt-% amount of isoparaffins in the catalytic cracking feedstock, which promotes the formation of propylene and C4 olefins, particularly propylene.

In certain embodiments, the catalytic cracking feedstock comprises aromatics less than 5 wt-%, preferably less than 1 wt-%, more preferably less than 0.5 wt-%, and naphthenes preferably less than 40 wt-%, or less than 30 wt-%, more preferably less than 20 wt-%, or less than 10 wt-%, even more preferably less than 1 wt-% of the total weight of the catalytic cracking feedstock. Aromatics do not form, or form only very little, propylene and C4 olefins and increase coke formation. A low content of naphthenes in the catalytic cracking feedstock is beneficial as naphthenes decrease formation of propylene and C4 olefins (compared to paraffins), and as naphthenes comprising a C6 ring are precursors for aromatics.

In certain embodiments, the hydrocarbon feed is a renewable hydrocarbon feed having a biogenic carbon content of at least 70 wt-%, preferably at least 80 wt-% or at least 90 wt-%, more preferably at least 95 wt-%, particularly preferably about 100 wt-% based on the total weight of carbon in the hydrocarbon feed as determined according to EN 16640 (2017). A renewable hydrocarbon feed provides an environmentally sustainable feedstock and yields environmentally sustainable cracking products. Particularly, a renewable hydrocarbon feed enables production of renewable (biogenic or bio-based) propylene and/or C4 olefins, which renewable propylene may be used e.g. in polymer production to produce renewable polymers.

Carbon atoms of renewable or biological origin (biogenic carbon) comprise a higher number of unstable radiocarbon (¹⁴C) atoms compared to carbon atoms of fossil origin. Therefore, it is possible to distinguish between carbon compounds derived from renewable or biological raw material and carbon compounds derived from fossil raw material by analysing the ratio of ¹²C and ¹⁴C isotopes. Thus, a particular ratio of said isotopes can be used as a “tag” to identify renewable carbon compounds and differentiate them from non-renewable carbon compounds. The isotope ratio does not change in the course of chemical reactions. Examples of a suitable method for analysing the content of carbon from biological or renewable sources are DIN 51637 (2014), ASTM D6866 (2020) and EN 16640 (2017). As used herein, the content of carbon from biological or renewable raw material is expressed as the biogenic carbon content meaning the amount of biogenic carbon in the material as a weight percent of the total carbon (TC) in the material as determined according to EN16640 (2017).

In the embodiments wherein the hydrocarbon feed is renewable, consequently also the cracking product is renewable, and the biogenic carbon content of the cracking product as a whole is essentially the same as that of the renewable hydrocarbon feed. Thus, if the hydrocarbon feed comprises biogenic carbon, also a fraction or fractions separated and/or recycled from the cracking product comprises biogenic carbon.

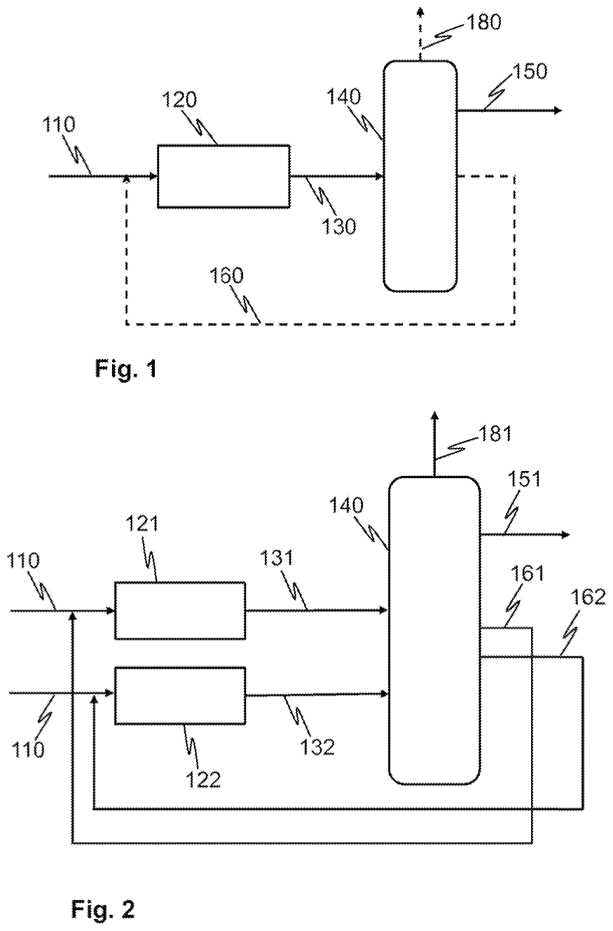

FIG. 1 shows a schematic drawing of a method according to an example embodiment. In the embodiment of FIG. 1, a catalytic cracking feedstock comprising a hydrocarbon feed 110 (fresh feed) and optionally a recycled fraction 160 is fed to a catalytic cracking reactor 120 comprising a solid catalyst for catalytic cracking. The solid catalyst may comprise zeolite or zeolite type material, wherein the micropore size of the solid catalyst is within a range from 4 to 6 Å, and wherein the solid catalyst preferably comprises strong acid sites.

Solid catalysts comprising zeolite or zeolite type material and having a micropore size within the range from 4 to 6 Å have surprisingly been found to promote the formation of olefins, including propylene and C4 olefins. Catalysts having strong acidity are beneficial, as they facilitate cracking of the feedstock at temperatures within the temperature range of the present method, namely from 300° C. to 450° C., with elevated conversion compared to catalysts having medium strong acidity. The somewhat lower conversion provided by catalysts with medium strong acidity may be compensated for example by increasing the catalyst amount and/or by using in combination with catalysts having strong acidity. On the other hand, catalysts having medium strong acidity may provide elevated selectivity to olefins, especially to ethylene, propylene and C4 olefins, compared to catalysts having strong acidity, and therefore it may be beneficial to use catalyst comprising zeolite or zeolite type material having medium strong acidity. The formation of propylene and C4 olefins, particularly propylene, is especially favoured as the catalytic cracking feedstock comprising the isoparaffin containing hydrocarbon feed and optional recycled fraction is cracked in the presence of such catalysts, also increasing the isoparaffin content of the catalytic cracking feedstock further favouring the formation of propylene and C4 olefins, particularly propylene. Advantageously, the solid catalyst comprises p zeolite, ZSM-5, SAPO-34 and/or MCM-22, preferably ZSM-5, MCM-22 and/or SAPO-34. ZSM-5, MCM-22 and SAPO-34 have been found to particularly promote the formation of propylene and C4 olefins. In addition to zeolite or zeolite type material, preferably comprising p zeolite, ZSM-5, SAPO-34 and/or MCM-22, more preferably ZSM-5, MCM-22 and/or SAPO-34, the solid catalyst may comprise a carrier, that may be catalytically active, such as silica-alumina or clay, or catalytically inactive, such as silica, to improve the mechanical durability and formability of the solid catalyst. The solid catalyst may comprise further additives such as a binder, or any other commonly known catalyst additive(s).

The acidic property of zeolites, including strength of acidity, may be determined by well known methods, for example by adsorption-desorption methods where release of adsorbed base substance like ammonia or pyridine at higher temperature indicates presence of strong acid sites. As one example of usable adsorption-desorption methods a temperature programmed desorption of ammonia can be mentioned, for examples as performed in accordance with the procedure described in Niwa et al (Niwa, M., Katada, N. Measurements of acidic property of zeolites by temperature programmed desorption of ammonia. Catalysis Surveys from Asia 1, 215-226 (1997)). Yet anotherwell known method fordetermining the acidic property of zeolites, including strength of acidity, is 1H-NMR method, for examples as performed in accordance with the procedure described in Heeribout et al (Heeribout L., Semmer V., Batamack P., Dorémieux-Morin C., Fraissard J. Brønsted acid strength of zeolites studied by ¹H NMR: scaling, influence of defects. Microporous and Mesoporous Materials, Volume 21, Issues 4-6, May 1998, Pages 565-570).

The fixed bed reactor may comprise one or more catalyst beds, the solid catalyst being comprised in at least one of said catalyst beds. For example, the fixed bed reactor may comprise two catalyst beds in series. In certain embodiments, the fixed bed reactor comprises a first catalyst bed comprising a first solid catalyst, the first solid catalyst preferably comprising a dehydrogenation agent, such as a metal, for activating long-chain (C16-C22) hydrocarbons or paraffins, followed by a second catalyst bed, the second catalyst bed preferably comprising a second solid catalyst preferably as described above, i.e. comprising zeolite or zeolite type material with 4-6 Å pore size and preferably having strong acidity for activating C6-C12 hydrocarbons or paraffins.

The catalytic cracking in the fixed bed reactor is preferably carried out in cycles, and a cycle is advantageously continued until the conversion decreases to an undesired level, for example below 0.10, or below 0.20. During a cracking cycle, the activity of the solid catalyst tends to decrease gradually due to fouling e.g. by coke deposited on the solid catalyst. Thus, the catalytic cracking in the fixed bed reactor is preferably carried out in cycles and the solid catalyst is advantageously regenerated between the cycles or between each cycle. Preferably, the catalytic cracking cycle lasts at least 24 hours. Preferably, the duration of the catalytic cracking cycle is extended by compensating for the gradual decrease in conversion due to catalyst fouling by increasing the temperature in the fixed bed reactor (reaction temperature). When the reaction temperature may not be increased anymore without undue negative impact on the catalytic cracking process and/or cracking product, and/or the conversion decreases below 0.20, or below 0.10, the solid catalyst is advantageously regenerated.

The moving solid catalyst reactor may be of any conventional type, such as a fluidized bed catalytic cracking reactor or a moving bed catalytic cracking reactor. The advantage of using a moving solid catalyst reactor is that it allows an excellent integration of the cracking reactor and a catalyst regenerator. In this way the catalyst may be regenerated continuously, and high thermal efficiency provided e.g. by burning off the coke in the regenerator providing the energy necessary for catalytic cracking without much loss. Since the catalytic cracking process using a moving solid catalyst is a continuous process, there is no need to take a reactor offline for regenerating the catalyst, thereby enhancing productivity.

The regeneration may be performed for example by flushing the solid catalyst with hot gas or by burning off deposited coke from the solid catalyst or by a combination thereof. Burning may be seen as an example of chemical regeneration by oxidation, in the presence of molecular oxygen or an oxygen-containing substance. Flushing may be seen as an example of physical regeneration, where adsorbed coke-precursors are desorbed using hot gas. Preferably, if the solid catalyst is regenerated by flushing occasionally, then from time to time burning will be used to regenerate the solid catalyst. In certain embodiments, the catalyst is first regenerated between several cracking cycles by flushing with hot gas, and then regenerated by burning off deposited coke. Flushing is beneficial in the sense that it allows recovering valuable renewable (bio-based) coke (when the hydrocarbon feed is a renewable hydrocarbon feed), that may then be further valorised by processing to shorter renewable (bio-based) hydrocarbons. Burning causes the renewable coke to be lost as carbon oxides, but on the other hand burning is beneficial in the sense that it is an efficient regeneration method, so that prolonged cracking cycles may be achieved after regeneration by burning compared to regeneration by flushing. Furthermore, when using moving solid catalyst reactor, burning off the coke provides thermal energy for the catalytic cracking.

The regeneration temperature is typically from 0° C. to 50° C. higher, preferably from 10° C. to 30° C. higher, than the highest catalytic cracking temperature (reaction temperature) used during cracking or during a cracking cycle. For example, burning off deposited coke may be carried out in the presence of molecular oxygen, e.g. by introducing air to a catalyst bed of the fixed bed reactor or to a separate catalyst regenerator, preferably at a temperature selected from a range from 400° C. to 550° C., preferably from 450° C. to 550° C.

In certain embodiments, the catalytic cracking may optionally be performed in two or more fixed bed reactors in parallel. In such embodiments, when a fixed bed reactor is being regenerated, catalytic cracking may continue simultaneously in another fixed bed reactor. This enables regeneration of the solid catalyst without having to shut down the entire catalytic cracking process or unit.

Preferably, the reaction conditions and/or the catalyst in the catalytic cracking reactor are selected so that the conversion of catalytic cracking feedstock in the catalytic cracking reaction is not too high. Advantageously, the conversion of catalytic cracking feedstock in the catalytic cracking reaction is within a range from 0.20 to 0.85, preferably from 0.20 to 0.80. A moderate conversion promotes a high conversion normalised yield of propylene, and it may also promote conversion normalised yield of C4 olefins. If the conversion is much lower than 0.20, then formation of propylene and C4 olefins may decrease whereas formation of C5-C10 hydrocarbons may increase, and more recycling of particularly C5-C10 hydrocarbons is then needed in order to obtain desired amounts of propylene and/or C4 olefins. Recycling may also become less economical. If the conversion is much higher than 0.85, then the conversion normalised yield of propylene and C4 olefins may start to decrease whereas formation of aromatics and naphthenes may start to increase and reach an undesired high level. A moderate conversion in the range from 0.20 to 0.85, preferably from 0.20 to 0.80, is particularly advantageous in embodiments wherein the catalytic cracking feedstock comprises a recycled fraction separated from the cracking product. Because the formation of propylene is promoted by a moderate conversion, a particularly high overall propylene yield is obtained from a given amount of hydrocarbon feed in embodiments wherein at least a portion of the cracking product is recycled back to the catalytic cracking feedstock for catalytic cracking.

In certain embodiments, the catalytic cracking is performed at a temperature selected from a range from 350° C. to 450° C. Temperatures selected from this range promote a high conversion normalised yield of propylene. In certain embodiments, the catalytic cracking is performed at a temperature selected from a range from 370° C. to 450° C., preferably from 400° C. to 450° C. Such somewhat higher temperature ranges may provide higher conversions especially in a once-through (i.e. no recycle) system. However, at reaction temperatures above 450° C. coke formation may start to increase and the formation of aromatics may start to increase such that an undesired high level of aromatics in the cracking effluent (cracking product) is reached. In certain embodiments, the catalytic cracking is performed at a temperature selected from a range from 350° C. to 430° C., preferably from 350° C. to 400° C., more preferably from 360° C. to 400° C. Such somewhat lower temperature ranges provide less coking and longer cycles as the solid catalyst is not fouled as quickly compared to higher reaction temperatures.

In certain embodiments, the weight hourly space velocity (WHSV, i.e. catalytic cracking feedstock mass flow/solid catalyst mass) of the catalytic cracking is selected from a range from 0.01 to 10, preferably from a range from 0.1 to 5. The unit of the WHSV is g catalytic cracking feedstock/g catalyst per hour. WHSV selected from these ranges promote a high conversion normalised yield of propylene. If the WHSV is much lower than 0.01 g catalytic cracking feedstock/g catalyst per hour, olefins may start to polymerize, and if the WHSV is much higher than 10, yields of the desired cracking products, such as propylene and C4 olefins, start to decrease.

In certain embodiments, the catalytic cracking is performed at a pressure selected from a range from 0.1 MPa to 2 MPa, preferably from 0.1 MPa to 1 MPa, more preferably from 0.15 MPa to 1 MPa, and even more preferably from 0.20 MPa to 1 MPa. Pressures selected from these ranges promote a high conversion normalised yield of propylene. A reaction pressure above atmospheric pressure, such as 0.15 MPa or higher, or 0.20 MPa or higher, may simplify the process allowing e.g. easier gas-liquid separation (compared to processes conducted at a lower pressure, such as atmospheric pressure or below).

In certain embodiments, the present method is carried out at an industrial scale. In industrial scale processes, it is often desired to keep the product stream of a process rather constant and to adjust the process parameters accordingly. For example, the conversion of the catalytic cracking feedstock on a fresh solid catalyst may differ from the conversion on a spent solid catalyst. The effect on the conversion of the gradual fouling or inactivation of the solid catalyst during catalytic cracking may be mitigated by adjusting the reaction conditions in the catalytic cracking reactor, especially in the fixed bed reactor, for example by increasing the reaction temperature.

It was found that in the present method particularly the temperature and the weight hourly space velocity (WHSV, i.e. catalytic cracking feedstock mass flow/solid catalyst mass) of the catalytic cracking controls the degree of conversion.

Accordingly, especially by adjusting the temperature and WHSV of the catalytic cracking, a moderate conversion promoting a high conversion normalised yield of propylene and C4 olefins, particularly propylene, can be achieved. Thus, in certain embodiments, the catalytic cracking is performed at a temperature selected from a range from 350° C. to 450° C., preferably from 350° C. to 400° C., more preferably the temperature is approximately 400° C., and at a weight hourly space velocity (WHSV, i.e. catalytic cracking feedstock mass flow/catalyst mass) selected from a range from 0.01 to 10, preferably from 0.1 to 5, more preferably from 0.5 to 3. The unit of the WHSV is g catalytic cracking feedstock/g catalyst per hour.

In the embodiment of FIG. 1, the cracking product 130 obtained by subjecting the catalytic cracking feedstock to catalytic cracking in the catalytic cracking reactor 120, i.e. the outflow or cracking effluent of the catalytic cracking reactor 120, is fed to a separation unit 140, for example a distillation unit, to separate from the cracking product at least a fraction 150 comprising propylene, C4 olefins, or both.

In certain other embodiments, the catalytic cracking reactor, especially a fixed bed reactor may be positioned within a distillation unit. For example, a fixed bed reactor and the distillation unit may form a catalytic distillation unit, wherein the method comprises subjecting the catalytic cracking feedstock to catalytic cracking in the fixed bed reactor within the distillation unit to form a cracking product, and separating from the cracking product by distillation in the distillation unit a fraction comprising propylene, C4 olefins, or both.

Optionally, the fraction comprising propylene, C4 olefins, or both may be subjected to further purification and/or fractionation steps. The optional purification and/or fractionation steps or treatments may be selected depending on the intended end use and/or desired degree of purity and/or target specification of the propylene and/or C4 olefins.

For example, the fraction comprising C4 olefins, or both propylene and C4 olefins, may be fractionated to separate from said fraction a certain C4 olefin or certain C4 olefins. In certain embodiments, the method comprises fractionating the fraction comprising C4 olefins, or both propylene and C4 olefins, to obtain one or more of 1-butene, trans-2-butene, cis-2-butene, butadiene, and isobutene as fractionated composition(s). Any conventional fractionation method suitable for fractionating C4 olefins may be used. The cracking product fraction comprising C4 olefins, or both propylene and C4 olefins, may be used as such, or after fractionation or purification, for producing alkylate, a high octane gasoline component, by reacting with isobutane.

In certain embodiments, the method comprises subjecting the fraction comprising propylene, or both propylene and C4 olefins to a purification treatment to obtain purified propylene. There are several types of conventional purification treatments or processes suitable for increasing propylene content of a product fraction and/or removing impurities of a fraction rich in propylene. For example, propane may be separated from propylene by distillation, commonly known as C3 splitter. A suitable purification treatment may be selected based on the intended purity grade and/or the intended end use of the propylene. A desired purity grade may be for example at least 50 wt-%, such as from 50 wt-% to 70 wt-%, propylene for refinery grade, or at least 90 wt-%, such as 90-95 wt-%, propylene for chemical grade, or at least 99 wt-%, such as at least 99.5 wt-%, propylene for polymer grade purity. As the propylene to total C3 weight ratio obtained using the present method is high, such as at least 0.7, less expensive equipment and energy may be required for achieving a desired higher purity grade.

Optionally, at least a portion of the fraction comprising propylene, C4 olefins, or both may be subjected to selective hydrotreatment for removing certain contaminants such as MAPD (propyne-propadiene mixture). However, an advantage of the present method is that the cracking product, and thus the fraction comprising propylene, C4 olefins, or both, may contain only very small amounts or be essentially free from for example propadiene, which could make selective hydrotreatment of the fraction in certain embodiments even redundant. MAPD (propyne-propadiene mixture) is harmful for the quality and further use of propylene compositions as well as compositions rich in one or more C4 olefin.

Optionally, at least a portion of the cracking product fraction comprising propylene, C4 olefins, or both may be contacted with an active material, such as an absorbent, an adsorbent, a purification catalyst, a reactant, a molecular sieve, or a combination thereof, for removing at least one of CO, CO₂, or C₂H₂. At least a portion of the cracking product fraction comprising propylene, C4 olefins, or both may optionally be passed through at least one purification train comprising active material, or at least one bed of active material. The contacting may be performed in a single vessel. Optionally, bringing at least a portion of the fraction comprising propylene, C4 olefins, or both into contact with active material may be performed in multiple vessels preferably connected in series, i.e. allowing the fraction to be purified to be passed from one vessel to the next for further purification. However, an advantage of the present method is that the cracking product, and thus the fraction comprising propylene, C4 olefins, or both, may contain only very small amounts or be essentially free from for example CO and CO₂, which could make in certain embodiments removal of CO and/or CO₂ from the fraction even redundant.

For example, the fraction comprising propylene may be a fraction of C3 hydrocarbons and/or the fraction comprising C4 olefins may be a fraction of C4 hydrocarbons. Thus, in certain embodiments, the method comprises separating from the cracking product a fraction of C3 hydrocarbons and/or a fraction of C4 hydrocarbons.

In certain other embodiments, the fraction comprising propylene, C4 olefins, or both is a fraction of C2-C4 hydrocarbons or a fraction of C3-C4 hydrocarbons. In embodiments wherein the fraction comprising propylene, C4 olefins, or both is a fraction of C2-C4 hydrocarbons or C3-C4 hydrocarbons, the method may comprise fractionating from the fraction of C2-C4 hydrocarbons or C3-C4 hydrocarbons a fraction of C3 hydrocarbons and/or a fraction of C4 hydrocarbons for example by distillation.

The method of the present disclosure enables production of propylene at least with refinery grade purity (such as at least 70 wt-% propylene) by separating a fraction of C3 hydrocarbons directly from the cracking product or from a fraction separated from the cracking product, such as the fraction of C2-C4 hydrocarbons, without subjecting said fraction of C3 hydrocarbons to further purification steps. Optionally, the fraction of C3 hydrocarbons may be subjected to purification step(s) or purification treatment(s) to obtain propylene with even higher purity, such as at least 90 wt-% or at least 99 wt-% propylene. As mentioned in the foregoing, there are several types of purification treatments, and a suitable purification treatment may be selected based on the intended purity grade and/or the intended end use of the propylene.

Optionally, further fractions may be separated and optionally recovered from the cracking product, for example a fraction of C5-C9 hydrocarbons.

In the embodiment of FIG. 1, a fraction 180 of methane and gasses lighter than methane is optionally separate from the cracking product. This fraction is typically burned to energy. Also, a fraction 160 of hydrocarbons having a carbon number of at least C5, preferably at least C10, may optionally be separated from the cracking product. At least a portion of the fraction of hydrocarbons having a carbon number of at least C5, preferably at least C10, is preferably recycled back to the catalytic cracking feedstock for catalytic cracking as the optional recycled fraction. The recycled fraction may be combined or mixed with the catalytic cracking feedstock before the catalytic cracking feedstock enters the catalytic cracking reactor or the recycled fraction may be fed to the catalytic cracking reactor wherein it forms a part of the catalytic cracking feedstock. Optionally, in embodiments wherein the fraction of hydrocarbons having a carbon number of at least C5, preferably at least C10, is separated from the cracking product, the method may comprise subjecting said fraction or at least a portion thereof to a purification treatment to obtain a purified fraction of hydrocarbons having a carbon number of at least C5, preferably at least C10, and recycling at least a portion of said purified fraction to the catalytic cracking feedstock. In the purification treatment, for example aromatics may be removed from at least a portion of the fraction of hydrocarbons having a carbon number of at least C5, preferably at least C10. In certain embodiments, at least a portion of the fraction of hydrocarbons having a carbon number of at least C5, preferably at least C10, is subjected to partial hydrotreatment, such as hydrogenation, to reduce or remove therefrom at least diolefins, and at least a portion of the partially hydrotreated fraction is recycled to the catalytic cracking feedstock. Diolefins tend to increase coke formation and fouling of the solid catalyst and may with other compounds also promote formation of explosive gums (NOx gums). In certain embodiments, at least a portion of the fraction of hydrocarbons having a carbon number of at least C5, preferably at least C10, is subjected both to partial hydrotreatment, such as hydrogenation, to reduce or remove therefrom at least diolefins, and to a purification treatment for example to remove aromatics, and preferably at least a portion of the purified and partially hydrotreated fraction of hydrocarbons having a carbon number of at least C5, preferably at least C10 is recycled back to the catalytic cracking feedstock.

In certain embodiments, the method comprises separating from the cracking product a fraction of C5-C9 hydrocarbons, and optionally recovering at least a portion of the fraction of C5-C9 hydrocarbons for use as a component in gasoline compositions and/or in chemical products such as in solvents, thinners and spot removers, and/or optionally recycling at least a portion of the fraction of C5-C9 hydrocarbons to the catalytic cracking feedstock. Preferably, after separating from the cracking product, the fraction of C5-C9 hydrocarbons is subjected to a hydrotreatment such as hydrogenation of olefins. Such optional hydrotreatment may reduce coke-formation when the fraction of C5-C9 hydrocarbons is used for recycling, or allow higher shares of the fraction to be incorporated in gasoline compositions and/or chemical products. Optionally, at least a portion of the fraction of C5-C9 hydrocarbons may be subjected to a purification treatment to remove therefrom at least aromatics to obtain a purified fraction of C5-C9 hydrocarbons, and preferably recycling at least a portion of the purified fraction of C5-C9 hydrocarbons to the catalytic cracking feedstock. This may be achieved e.g. by hydrodearomatization, solvent extraction, or any other known method. Aromatics, such as benzene, xylene and/or toluene, removed from at least a portion of the fraction of C5-C9 hydrocarbons in the optional purification step, may be recovered and provided for value added use. In certain embodiments, at least a portion of the fraction of C5-C9 hydrocarbons is subjected to both the optional hydrotreatment, such as hydrogenation of olefins, and the optional purification treatment to remove at least aromatics, and preferably at least a portion of the hydrotreated and purified fraction of C5-C9 hydrocarbons is recycled back to the catalytic cracking feedstock.

A recycled fraction having similar carbon chain lengths than the fresh feed (hydrocarbon feed) is advantageous, as it provides a rather uniform catalytic cracking feedstock allowing adjustment or controlling of catalytic cracking conditions to promote formation of propylene and/or C4 olefins. It may also reduce variation in the cracking effluent, and it is expected to blend well with the fresh feed.

In certain embodiments, the catalytic cracking is performed without feeding (additional) molecular hydrogen (H₂) to the catalytic cracking reactor. The method may thus be carried out without providing a (fresh) feed of molecular hydrogen (H₂) to the catalytic cracking reactor. Because the method of the present disclosure is a catalytic cracking method for producing olefins, there is no need to feed (additional) molecular hydrogen to the catalytic cracking reactor. Preferably, both the hydrocarbon feed and the optional recycled fraction are essentially free from molecular hydrogen (H₂).

FIG. 2 shows a schematic drawing of a method according to an example embodiment. In the embodiment of FIG. 2, a first catalytic cracking feedstock comprising hydrocarbon feed 110 (fresh feed) and a first recycled fraction 161 is fed to a first catalytic cracking reactor 121 comprising a solid catalyst for catalytic cracking; and a second catalytic cracking feedstock comprising hydrocarbon feed 110 (fresh feed) and a second recycled fraction 162 is fed to a second catalytic cracking reactor 122 comprising a solid catalyst for catalytic cracking. The first and/or second catalytic cracking reactors may be catalytic cracking reactors as described above, such as fixed bed reactors. The solid catalyst(s) may be a solid catalyst as described above. Optionally, when the catalytic cracking reactors 121 and 122 are fixed bed reactors, the first catalytic cracking feedstock may be fed to at least one further fixed bed reactor in parallel with the first fixed bed reactor and the second catalytic cracking feedstock may be fed to at least one further fixed bed reactor in parallel with the second fixed bed reactor, respectively, allowing regeneration of the solid catalyst in the first and/or the second fixed bed reactor while simultaneously continuing the catalytic cracking.

In the embodiment of FIG. 2, a first cracking product 131 obtained by subjecting the first catalytic cracking feedstock to catalytic cracking in the first catalytic cracking reactor 121, i.e. the outflow or effluent of the first catalytic cracking reactor 121, and a second cracking product 132 obtained by subjecting the second catalytic cracking feedstock to catalytic cracking in the second catalytic cracking reactor 122, i.e. the outflow or effluent of the second catalytic cracking reactor 122, are fed to a separation unit 140, such as a distillation unit, for separation of hydrocarbon fractions from the cracking products 131, 132. Alternatively, in certain other embodiments, the first cracking product and the second cracking product may be fed to respective separation units, i.e. the first cracking product is fed to a first separation unit for separation of hydrocarbon fractions from the first cracking product and the second cracking product is fed to a second separation unit for separation of hydrocarbon fractions from the second cracking product. The separation unit or distillation unit may be a catalytic distillation unit.

In the embodiment of FIG. 2, a fraction 151 comprising propylene, C4 olefins, or both, is separated from the cracking products 131, 132. Optionally, the fraction comprising propylene, C4 olefins, or both may be subjected to further purification and/or fractionation steps as described more in detail above. Further, in the embodiment of FIG. 2, a fraction comprising methane and gases lighter than methane 181 is separated from the cracking products 131, 132 and optionally burned to energy. A fraction of C5-C9 hydrocarbons 161 is in the embodiment of FIG. 2 separated from the cracking products 131, 132. The fraction of C5-C9 hydrocarbons or a portion thereof may optionally be subjected to a purification treatment to obtain a purified fraction of C5-C9 hydrocarbons. Preferably, at least aromatics are removed from at least a portion of the fraction of C5-C9 hydrocarbons in the optional purification treatment. In the embodiment of FIG. 2, at least a portion of the fraction of C5-C9 hydrocarbons or the purified fraction of C5-C9 hydrocarbons is recycled to the first catalytic cracking feedstock for catalytic cracking in the first catalytic cracking reactor 121. In the embodiment of FIG. 2, a fraction 162 of hydrocarbons having a carbon number of at least C10 is separated from the cracking products 131, 132. Optionally, the fraction of hydrocarbons having a carbon number of at least C10 or a portion thereof may be subjected to a purification treatment to obtain a purified fraction of hydrocarbons having a carbon number of at least C10. Although metals tend to get stuck on the solid catalyst, it is believed that for example metal impurities getting through tend to accumulate in the heavier fraction of hydrocarbons having a carbon number of at least C10, or at least C22. In the embodiment of FIG. 2, at least a portion of the fraction of hydrocarbons having a carbon number of at least C10 or the purified fraction of hydrocarbons having a carbon number of at least C10 is recycled to the second catalytic cracking feedstock for catalytic cracking in the second catalytic cracking reactor 122. Most aromatic compounds (benzene C6, toluene C7 and xylene C8) of the cracking products 131, 132 tend to be in the fraction of C5-C9 hydrocarbons.

Optionally, at least a portion of the fraction of C5-C9 hydrocarbons, or at least a portion of the purified fraction of C5-C9 hydrocarbons may be partially hydrotreated, such as hydrogenated, to reduce or remove therefrom at least diolefins, and at least a portion of the partially hydrotreated fraction of C5-C9 hydrocarbons may be recycled to the catalytic cracking feedstock. Similarly, at least a portion of the fraction of hydrocarbons having a carbon number of at least C10, or at least a portion of the purified fraction of hydrocarbons having a carbon number of at least C10 may be partially hydrotreated, such as hydrogenated, to reduce or remove therefrom at least diolefins, and at least a portion of the partially hydrotreated fraction of hydrocarbons having carbon number of at least C10 may be recycled to the catalytic cracking feedstock.

Recycling at least a portion of the fraction of C5-C9 hydrocarbons and at least a portion of the fraction of hydrocarbons having a carbon number of at least C10 separately from each other to respective catalytic cracking feedstocks for catalytic cracking in respective catalytic cracking reactors facilitates the recycling process, as for example pumping of the fractions (or portions of fractions) can be optimised. Additionally, the line for recycling C5-C9 does not require heating. The reaction conditions and/or catalysts in the first catalytic cracking reactor 121 and in the second catalytic cracking reactor 122 may differ from each other. This allows selecting the reaction conditions and/or catalysts so that the formation of propylene and/or C4 olefins may be promoted in both catalytic cracking reactors taking into account the different compositions of the recycled fractions.

Accordingly, in certain embodiments, the method comprises providing a first catalytic cracking feedstock comprising a hydrocarbon feed and a recycled fraction of C5-C9 hydrocarbons separated from the cracking product and optionally subjected to a purification treatment to remove at least aromatics from said fraction; and providing a second catalytic cracking feedstock comprising a hydrocarbon feed and a recycled fraction of hydrocarbons having a carbon number of at least C10 separated from the cracking product and optionally subjected to a purification treatment to remove for example aromatics and/or metals from said fraction; and catalytically cracking the first catalytic cracking feedstock in a first catalytic cracking reactor to obtain a first cracking product; and catalytically cracking the second catalytic cracking feedstock in a second catalytic cracking reactor to obtain a second cracking product; and separating from the first and the second cracking product at least a fraction comprising propylene, C4 olefins, or both. Reaction conditions, such as temperature, pressure and WHSV, and/or the catalysts in the first catalytic cracking reactor and in the second catalytic cracking reactor may be adjusted (essentially independently of each other) so that the coke formation remains in each catalytic cracking reactor on a desired low level and at the same time conversion is maintained between 0.20-0.85. The temperature, pressure and WHSV for achieving this may depend e.g. on the composition of the catalytic cracking feedstock, and in embodiments wherein the catalytic cracking reactor is a fixed bed catalytic cracking reactor, the stage of the cracking cycle (when conversion starts to decrease due to fouling of the solid catalyst, increasing the reaction temperature may be used to compensate for it).

The hydrocarbon feed of the present disclosure may for example be a hydrocarbon feed obtained through hydrotreatment comprising deoxygenation and isomerisation reactions of renewable oxygen containing hydrocarbons, a hydrocarbon feed obtained through a gas-to-liquid (GTL) process, such as a Fischer-Tropsch process, or a mixture thereof. Hydrocarbon compositions manufactured through gas-to-liquid (GTL) processes are characterized by paraffinic hydrocarbons having a broad carbon number distribution typically in the range of carbon numbers C9-C50, particularly C9-C24, and they may be subjected to an isomerisation treatment. Preferably, the hydrocarbon feed is obtained through hydrotreatment comprising deoxygenation and isomerisation reactions of renewable oxygen containing hydrocarbons. Oxygen containing hydrocarbons refer herein to organic molecules of carbon, hydrogen, and oxygen. Typically, the carbon number range of paraffinic hydrocarbons obtained through hydrotreatment comprising deoxygenation and isomerisation reactions of renewable oxygen containing hydrocarbons, such as renewable oils and/or fats, is narrower than that of paraffinic hydrocarbons obtained through GTL processes. Typically, paraffinic hydrocarbons obtained through hydrotreatment comprising deoxygenation and isomerisation reactions of renewable oxygen containing hydrocarbons comprise mainly compounds in the C14-C18 carbon number range.

In certain embodiments, the hydrocarbon feed is obtained by a process comprising: hydrotreating renewable oxygen containing hydrocarbons, preferably comprising one or more of fatty acids, fatty acid esters, resin acids, resin acid esters, sterols, fatty alcohols, oxygenated terpenes, and other renewable organic acids, ketones, alcohols, and anhydrides, preferably originating from vegetable oils, animal fats, microbial oils, or a combination thereof, the hydrotreating comprising deoxygenation and isomerisation reactions, to obtain a hydrotreatment product comprising isoparaffins, and removing vapor phase from the hydrotreatment product to obtain a vapour depleted hydrotreatment product and optionally recovering from the vapour depleted hydrotreatment product as the hydrocarbon feed a fraction comprising at least 50 wt-%, preferably at least 60 wt-%, further preferably at least 70 wt-%, more preferably at least 80 wt-%, and even more preferably at least 90 wt-% hydrocarbons having a carbon number of at least C10. The removed vapour phase may comprise H₂, CO, CO₂, H₂S, NH₃, H₂O, and/or C1-C4 hydrocarbons. Removing vapour phase may comprise or consist essentially of removing gaseous compounds (gaseous at NTP) and water from the hydrotreatment product. Preferably, the vapour depleted hydrotreatment product comprises less than 1 wt-% of gaseous compounds (NTP) and water. Gaseous compounds (NTP) refer herein to compounds that are in gas form under normal temperature and pressure, i.e. 20° C. and an absolute pressure of 1 atm (101.325 kPa). Gaseous compounds in the hydrocarbon feed may reduce yield of desired cracking products, and for example CO and CO₂ may cause product quality issues in the lighter cracking product fractions, such as fraction of or comprising C3 hydrocarbons.

In certain embodiments removing vapour phase from the hydrotreatment product is conducted by subjecting the hydrotreatment product to a gas-liquid separation. The gas-liquid separation may be conducted as a separate step (e.g. after the hydrotreatment product has left the hydrotreatment reactor or reaction zone) and/or as an integral step of the hydrotreatment step, e.g. within the hydrotreatment reactor or reaction zone. Majority of water that may form e.g. during hydrodeoxygenation, and potentially carried-over from the fresh renewable oxygen containing hydrocarbons, may be removed for example via a water boot in the gas-liquid separation step.

In certain embodiments the gas-liquid separation is carried out at a temperature selected from a range from 0° C. to 500° C., such as from 15° C. to 300° C., or from 15° C. to 150° C., preferably from 15° C. to 65° C., such as from 20° C. to 60° C., and preferably at the same pressure as that of the hydrotreatment step. In general, the pressure in the gas-liquid separation step may be within a range from 1 to 200 bar gauge, preferably from 10 to 100 bar gauge, or from 30 to 70 bar gauge.

Hydrotreated renewable (bio-based) hydrocarbon feeds comprising specified amounts of hydrocarbons having carbon numbers within specified carbon number ranges are obtainable e.g. by subjecting the hydrotreatment product and/or the vapour depleted hydrotreatment product preferably containing less than 1 wt-% of gaseous compounds (NTP) to a fractionation.

Most renewable raw material comprises materials having a high oxygen content. The renewable oxygen containing hydrocarbons may include one or more of fatty acids, whether in free or salt form; fatty acid esters, such as mono-, di- and triglycerides, alkyl esters such as methyl or ethyl esters, etc; resin acids, whether in free or salt form; resin acid esters, such as alkyl esters, sterol esters etc; sterols; fatty alcohols; oxygenated terpenes; and other renewable organic acids, ketones, alcohols, and anhydrides. Preferably the renewable oxygen containing hydrocarbons originate from one or more of vegetable oils, such as rapeseed oil, canola oil, soybean oil, coconut oil, sunflower oil, palm oil, palm kernel oil, peanut oil, linseed oil, sesame oil, maize oil, poppy seed oil, cottonseed oil, soy oil, tall oil, corn oil, castor oil, jatropha oil, jojoba oil, olive oil, flaxseed oil, camelina oil, safflower oil, babassu oil, seed oil of any of Brassica species or subspecies, such as Brassica carinata seed oil, Brassica juncea seed oil, Brassica oleracea seed oil, Brassica nigra seed oil, Brassica napus seed oil, Brassica rapa seed oil, Brassica hirta seed oil and Brassica alba seed oil, and rice bran oil, or fractions or residues of said vegetable oils such as palm olein, palm stearin, palm fatty acid distillate (PFAD), purified tall oil, tall oil fatty acids, tall oil resin acids, distilled tall oil, tall oil unsaponifiables, tall oil pitch (TOP), and used cooking oil preferably of vegetable origin; animal fats, such as tallow, lard, yellow grease, brown grease, fish fat, poultry fat, and used cooking oil of animal origin, microbial oils, such as algal lipids, fungal lipids and bacterial lipids.

Hydrotreatment of the oxygen containing hydrocarbons may involve various reactions where molecular hydrogen reacts with other components, or components undergo molecular conversions in presence of molecular hydrogen and a catalyst. The reactions include but are not limited to hydrogenation, hydrodeoxygenation, hydrodesulphurization, hydrodenitrogenation, hydrodemetallization, hydrocracking, hydropolishing, hydroisomerisation and hydrodearomatization.

Deoxygenation refers herein to removal of oxygen as H₂O, CO₂ and/or CO from the oxygen containing hydrocarbons by hydrodeoxygenation, decarboxylation and/or decarbonylation. Preferably the hydrotreatment comprises deoxygenation by hydrodeoxygenation (HDO) reactions and isomerisation by hydroisomerisation reactions. Hydrodeoxygenation refers herein to removal of oxygen as H₂O from oxygen containing hydrocarbons by means of molecular hydrogen under influence of a catalyst to obtain hydrocarbons, while hydroisomerisation means formation of branches to hydrocarbons by means of molecular hydrogen under influence of a catalyst that can be same or different as for HDO.

The hydrotreatment comprising deoxygenation and isomerisation reactions may be conducted in a single reactor conducting deoxygenation and isomerisation reactions in same or subsequent catalyst beds, or in separate reactors. Preferably the deoxygenation and isomerisation reactions of the hydrotreatment are conducted in separate deoxygenation and isomerisation steps in subsequent catalyst beds in a same reactor or in separate reactors.

Reaction conditions and catalysts suitable for the hydrodeoxygenation and isomerisation of renewable oxygen containing hydrocarbons, such as fatty acids and/or fatty acid derivatives, are known. Examples of such processes are presented in WO 2015/101837 A2, paragraphs [0032]-[0037], F1100248, Examples 1-3, and EP 1741768 A1, paragraphs [0038]-[0070], particularly paragraphs [0056]-[0070], and Examples 1-6. Also other methods may be employed, particularly another BTL (Biomass-To-Liquid) method may be chosen.

The hydrodeoxygenation of renewable oxygen containing hydrocarbons is preferably performed at a pressure selected from a range from 2 MPa to 15 MPa, preferably from 3 MPa to 10 MPa, and at a temperature selected from a range from 200 to 500° C., preferably from 280 to 400° C. The hydrodeoxygenation may be performed in the presence of known hydrodeoxygenation catalyst containing metal(s) from Group VIII and/or Group VIB of the Periodic System. The catalyst may be supported on any suitable support, such as alumina, silica, zirconia, titania, amorphous carbon, molecular sieves or combinations thereof. Preferably, the hydrodeoxygenation catalyst is supported Pd, Pt, Ni, or NiW catalyst, or supported Mo containing catalyst, such as NiMo or CoMo, catalyst, wherein the support is alumina and/or silica, or a combination of these catalysts. Typically, NiMo/Al₂O₃ and/or CoMo/Al₂O₃ catalysts are used.

The hydrodeoxygenation (HDO) of renewable oxygen containing hydrocarbons is preferably carried out in the presence of sulphided NiMo or sulphided CoMo catalysts in the presence of hydrogen gas. The HDO may be performed under a hydrogen pressure selected from a range from 1 MPa to 20 MPa, at temperatures selected from a range from 200° C. to 400° C., and liquid hourly space velocities selected from a range from 0.2 h⁻¹ to 10 h⁻¹. Using a sulfided catalyst, the sulfided state of the catalyst may be maintained during the HDO step by the addition of sulphur in the gas phase or by using a feedstock having sulphur containing mineral oil blended with the renewable oxygen containing hydrocarbons. The sulphur content of the total feedstock being subjected to hydrodeoxygenation may be, for example, within a range from 50 wppm (ppm by weight) to 20 000 wppm, preferably within a range from 100 wppm to 1000 wppm. Effective conditions for hydrodeoxygenation may reduce the oxygen content of the renewable oxygen containing hydrocarbons, such as fatty acids or fatty acid derivatives, to less than 1 wt-%, such as less than 0.5 wt-% or less than 0.2 wt-%.

The isomerisation is not particularly limited and any suitable approach resulting in isomerisation reactions may be used. However, catalytic hydroisomerisation treatments are preferred. The isomerisation treatment is preferably performed at a temperature selected from a range from 200° C. to 500° C., preferably from 280° C. to 400° C., such as from 300° C. to 350° C., and at a pressure selected from a range from 1 MPa to 15 MPa, preferably from 3 MPa to 10 MPa. The isomerisation treatment may be performed in the presence of known isomerisation catalysts, for example, catalysts containing a molecular sieve and/or a metal selected from Group VIII of the Periodic System and a support. Preferably, the isomerisation catalyst is a catalyst containing SAPO-11 or SAPO-41 or ZSM-22 or ZSM-23 or ferrierite and Pt, Pd, or Ni and Al₂O₃ or SiO₂. Typical isomerisation catalysts are, for example, Pt/SAPO-11/Al₂O₃, Pt/ZSM-22/Al₂O₃, Pt/ZSM-23/Al₂O₃ and/or Pt/SAPO-11/SiO2. The catalysts may be used alone or in combination. Catalyst deactivation during the isomerisation treatment may be reduced by the presence of molecular hydrogen in the isomerisation treatment. In certain preferred embodiments, the isomerisation catalyst is a noble metal bifunctional catalyst, such as Pt-SAPO and/or Pt-ZSM catalyst, which is used in combination with hydrogen.

The isomerisation reactions serve to isomerise at least part of the n-paraffins obtained through deoxygenation of renewable oxygen containing hydrocarbons. The isomerisation may comprise intermediate steps such as a purification step and/or a fractionation step.

The deoxygenation and isomerisation reactions may be performed either simultaneously or in sequence. In certain embodiments, obtaining the hydrocarbon feed comprises carrying out hydrodeoxygenation and hydroisomerisation reactions in a single step on the same catalyst bed using a single catalyst for this combined step, e.g. NiW, or a Pt catalyst, such as Pt/SAPO in a mixture with a Mo catalyst on a support, e.g. NiMo on alumina. Preferably, in embodiments where deoxygenation and isomerisation are performed in sequence, the deoxygenation is followed by the isomerisation.

The renewable oxygen containing hydrocarbons are preferably obtainable or derivable, or originating from plants and/or animals, including renewable oxygen containing hydrocarbons obtainable, derivable, or originating from fungi and/or algae and gene manipulated plants and/or animals. Renewable oxygen containing hydrocarbons may also be referred to as biological oxygen containing hydrocarbons, bio-based oxygen containing hydrocarbons, or biogenic oxygen containing hydrocarbons.

Fossil raw material or mineral raw material refer in the context of this disclosure to naturally occurring non-renewable compositions, such as crude oil, petroleum oil/gas, shale oil/gas, natural gas, or coal deposits, and the like, and combinations thereof, including any hydrocarbon-rich deposits that can be utilized from ground/underground sources. The term fossil or mineral may also refer to recycling material originating from non-renewable sources.

Typically, a hydrocarbon feed obtained from renewable oxygen containing hydrocarbons has a biogenic carbon content of at least 90 wt-%, preferably at least 95 wt-%, more preferably about 100 wt-% based on the total weight of carbon (TC) in the hydrocarbon feed as measured according to EN 16640 (2017). Typically, hydrocarbons derived from fossil crude oil based mineral oil have a biogenic carbon content of about 0 wt-%. The renewable oxygen containing hydrocarbons have preferably a biogenic carbon content of at least 90 wt-%, preferably at least 95 wt-%, more preferably about 100 wt-% based on the total weight of carbon (TC) in the renewable oxygen containing hydrocarbons as measured according to EN 16640 (2017).

Renewable oxygen containing hydrocarbons originating from renewable oils and/or fats typically comprise C10-C24 fatty acids and derivatives thereof, including esters of fatty acids, glycerides, i.e. glycerol esters of fatty acids. The glycerides may specifically include monoglycerides, diglycerides and triglycerides. Optionally, the renewable oxygen containing hydrocarbons may be at least partially derived or obtained from recyclable waste and/or recyclable residue, such as used cooking oil, free fatty acids, palm oil by-products or process side streams, sludge, side streams from vegetable oil processing, or a combination thereof.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

Four renewable hydrocarbon feed samples (P1, P2, P3, P4) with different isomerisation degrees were provided by catalytic hydrotreatment, involving hydrodeoxygenation and isomerisation reactions, of renewable oxygen containing hydrocarbons originating from animal fat and vegetable oils. The hydrotreatment conditions were varied to provide the renewable hydrocarbon feed samples with different isoparaffin contents. The hydrotreatment products were degassed to remove gaseous compounds (NTP), and water vapour, and the liquid hydrotreatment products were fractionated by distillation collecting distillation cuts having boiling ranges (initial boiling points (IBP) and final boiling points (FBP)) as reported in Table 2. The biogenic carbon content of each of P1-P4 was approximately 100 wt-%, based on the total weight of carbon in the respective hydrocarbon feed sample, as measured according to EN 16640 (2017).

### Characteristics of Hydrocarbon Feed Samples

Cloud point and density of P1, P2, P3, and P4 are shown in Table 1, and distillation characteristics of P1, P2, P3, and P4 are shown in Table 2.

TABLE 1
Could point and density of hydrocarbon feed samples P1-P4

| Parameter | Method | P1 | P2 | P3 | P4 |
|---|---|---|---|---|---|
| Cloud Point (° C.) | ASTMD7689-17 | 23.1 | −2 | −36 | −30.1 |
| Density at 15° C. | ENISO12185: 1996 | 793.7 | 779.1 | 779.0 | 788.1 |
| (kg/m³) | | | | | |

TABLE 2
Distillation characteristics of hydrocarbon feed samples
P1-P4 determined according to ENISO3405: 2019.

| Property | P1 | P2 | P3 | P4 |
|---|---|---|---|---|
| DIS-IBP (° C.) | 273 | 194.45 | 177.9 | 284.1 |
| DIS-05 (° C.) | 288.7 | 267.3 | 244.5 | 292.2 |
| DIS-10 (° C.) | 290.6 | 272.5 | 259.4 | 292.7 |
| DIS-20 (° C.) | 292.3 | 277.3 | 269.4 | 293.1 |
| DIS-30 (° C.) | 293.7 | 279.45 | 273.5 | 293.6 |
| DIS-40 (° C.) | 295.2 | 281.45 | 276.2 | 294.1 |
| DIS-50 (° C.) | 296.6 | 283.05 | 278.4 | 294.6 |
| DIS-60 (° C.) | 298 | 285 | 280.4 | 295.2 |
| DIS-70 (° C.) | 299.6 | 287.35 | 282.9 | 295.9 |
| DIS-80 (° C.) | 301.5 | 290.6 | 285.9 | 297.3 |
| DIS-90 (° C.) | 303.9 | 293.2 | 289.6 | 300.2 |
| DIS-95 (° C.) | 307.3 | 297.9 | 294.9 | 308.2 |
| DIS-FBP (° C.) | 315.1 | 304.6 | 307.8 | 326.9 |
| DIS-LOSS (vol-%) | | 0.8 | 0.5 | 0.2 |
| DIS-RECOVERY (vol-%) | 97.9 | 97.9 | 98.1 | 97.6 |
| DIS-RESIDUE (vol-%) | 1 | 1.3 | 1.4 | 2.2 |

### Measurement of Isomerisation Degree

The hydrocarbon feed samples, namely P1, P2, P3 and P4, were analysed by gas chromatography (GC). The samples were analysed as such, without any pretreatment. The method is suitable for hydrocarbons C2-C36. N-paraffins and groups of isoparaffins (C1-, C2-, C3-substituted and C3-substituted) were identified using mass spectrometry and a mixture of known n-paraffins in the range of C2-C36. The chromatograms were split into three groups of paraffins (C1-, C2-/C3- and ≥C3-substituted isoparaffins/n-paraffin) by integrating the groups into the chromatogram baseline right after n-paraffin peak. N-paraffins were separated from ≥C3-substituted isoparaffins by integrating the n-alkane peak tangentially from valley to valley and compounds or compound groups were quantified by normalisation using relative response factor of 1.0 to all hydrocarbons. The limit of quantitation for individual compounds was 0.01 wt-%. Settings of the GC are shown in Table 3.

TABLE 3
Settings of GC determination of n- and i-paraffins.

| | GC |
|---|---|
| Injection | split/splitless-injector |
| | Split 80:1 (injection volume 0.2 μL) |
| Column | DB ™-5 (length 30 m, i.d. 0.25 m, phase thickness |
| | 0.25 μm) |
| Carrier gas | He |
| Detector | FID (flame ionization detector) |
| GC program | 30° C. (2 min)-5° C./min-300° C. |
| | (30 min), constant flow 1.1 mL/min) |

The wt-% amount of n-paraffins and the wt-% amount of (total) i-paraffins, based on the total weight of the analysed hydrocarbon feed sample, were determined for each of P1-P4, and are shown in Table 4. Table 4 also shows carbon chain lengths of paraffins (n-paraffins and i-paraffins) in the samples.

TABLE 4
n-paraffin and i-paraffin contents
of hydrocarbon feed samples P1-P4

| | P1 | | P2 | | P3 | | P4 | |
|---|---|---|---|---|---|---|---|---|
| C. | | iP | | iP | | iP | | iP |
| No | nP | total | nP | total | nP | total | nP | total |
| 2 | 0 | 0 | 0 | 0 | 0 | 0 | 0 | 0 |
| 3 | 0 | 0 | 0 | 0 | 0 | 0 | 0 | 0 |
| 4 | 0 | 0 | 0 | 0 | 0.01 | 0 | 0 | 0 |
| 5 | 0.02 | 0.02 | 0 | 0 | 0.02 | 0.01 | 0 | 0 |
| 6 | 0.02 | 0.03 | 0.06 | 0.03 | 0.05 | 0.04 | 0 | 0 |
| 7 | 0.02 | 0.04 | 0.14 | 0.21 | 0.09 | 0.12 | 0 | 0 |
| 8 | 0.02 | 0.04 | 0.14 | 0.23 | 0.26 | 0.51 | 0 | 0 |
| 9 | 0.02 | 0.06 | 0.16 | 0.27 | 0.23 | 0.76 | 0 | 0 |
| 10 | 0.05 | 0.04 | 0.15 | 0.3 | 0.19 | 0.91 | 0 | 0 |
| 11 | 0.04 | 0.02 | 0.15 | 0.29 | 0.15 | 0.93 | 0 | 0 |
| 12 | 0.09 | 0.04 | 0.19 | 0.31 | 0.13 | 1.08 | 0 | 0 |
| 13 | 0.27 | 0.06 | 0.25 | 0.39 | 0.11 | 1.12 | 0 | 0 |
| 14 | 1.01 | 0.13 | 0.43 | 0.65 | 0.35 | 1.73 | 0 | 0 |
| 15 | 4.30 | 0.42 | 5.57 | 8.2 | 1.53 | 9.88 | 0.03 | 0.19 |
| 16 | 15.95 | 1.20 | 9.58 | 18.85 | 1.6 | 26.6 | 0.56 | 1.86 |
| 17 | 15.92 | 1.56 | 5.26 | 13.27 | 1.88 | 15.4 | 2.05 | 20.31 |
| 18 | 52.33 | 3.43 | 8.73 | 24.94 | 0.79 | 31.77 | 1.85 | 67.82 |
| 19 | 0.55 | 0.24 | 0.06 | 0.3 | 0.04 | 0.47 | 0.18 | 2.71 |
| 20 | 1.04 | 0.09 | 0.06 | 0.31 | 0.02 | 0.39 | 0.03 | 1.66 |
| 21 | 0.08 | 0.03 | 0.01 | 0.04 | 0.01 | 0.11 | 0.02 | 0.34 |
| 22≤ | 0.27 | 0.53 | 0.03 | 0.43 | 0.03 | 0.7 | 0.00 | 0.36 |
| Total | 92.00 | 8.00 | 30.96 | 69.04 | 7.48 | 92.52 | 4.72 | 95.25 |

As can be seen from Table 4, the hydrocarbon feed samples P1-P4 were highly paraffinic, and contained from about 8 to about 95 wt-% isoparaffins, based on the total weight of the corresponding hydrocarbon feed sample. The hydrocarbon feed samples contained, based on the total weight of the corresponding hydrocarbon feed sample, hydrocarbons having a carbon number of at least C10 as follows: P1 about 100 wt-%, P2 about 99 wt-%, P3 about 98 wt-% and P4 about 100 wt-%; and C14-C18 hydrocarbons as follows: P1 about 96 wt-%, P2 about 95 wt-%, P3 about 92 wt-% and P4 about 95 wt-%.

### Fixed Bed Catalytic Cracking of Hydrocarbon Feed

Experiments were carried out with a continuous-flow fixed-bed microreactor with a reactor tube of about 200 ml in volume. The microreactor setup contained feed tanks, inlets for air, He, N₂ and H₂, the reactor, and a product tank followed by a gas trap. The reactor tube was heated with an oven with a maximum operating temperature set to 500° C. A gas operated pump was used to pump hydrocarbon feed from two feed tanks.

The catalyst material was ZSM-5. Catalysts were prepared by calcination, and pelletized, crushed and sieved to obtain a desired particle size.

For every test run, the reactor was packed with inert SiC 30 as a filler for ensuring good heat balance and trickle flow of the reactant (catalytic cracking feedstock) inside the reactor, with the catalyst placed between two SiC 30 beds (thus forming a fixed catalyst bed), approximately in the middle of the reactor. This region approximately in the middle of the reactor is mostly isothermal, providing even temperature across the catalyst bed. 30 g of catalysts were used in each catalytic cracking experiment. Glass beads were used at the top of the reactor to ensure even distribution of the hydrocarbon feed sample inside the reactor. Glass wool was used at the ends of the reactor and between each layer for ensuring that the packing remained in place.

The conditions during fixed bed catalytic cracking test runs using hydrocarbon feed samples P1-P4 respectively as catalytic cracking feedstock are presented in Table 5. The temperatures in Table 5 are reaction temperatures, and WHSVs are expressed as mass flow of the hydrocarbon feed/catalyst mass.

TABLE 5
Catalytic cracking conditions for hydrocarbon feed samples P1-P4

| | P1 | P2 | P3 | P4 |
|---|---|---|---|---|
| | (8 wt-% | (69 wt-% | (92.5 wt-% | (95.25 wt-% |
| | iP) | iP) | iP) | iP) |
| T (° C.) | 400 | 400 | 400 | 400 |
| p (MPa) | 0.1 | 0.1 | 0.1 | 0.1 |
| WHSV (g catalytic | 0.6 | 0.6 | 0.6 | 0.6 |
| cracking feedstock/g | | | | |
| catalyst per hour) | | | | |

### Results—Fixed Bed Catalytic Cracking of Hydrocarbon Feed

The results of the fixed bed catalytic cracking test runs using hydrocarbon feed samples P1-P4 respectively as catalytic cracking feedstock are presented in Tables 6-9.

TABLE 6
Catalytic cracking product yields
for hydrocarbon feed samples P1-P4

| | (mol-%) | | | |
|---|---|---|---|---|
| | P1 | P2 | P3 | P4 |
| | (8 wt-% | (69 wt-% | (92.5 wt-% | (95.25 wt-% |
| | iP) | iP) | iP) | iP) |
| Conversion | 31 | 32 | 27 | 29 |
| H₂ | 0.8 | 0.9 | 1.3 | 1.7 |
| methane | 0.1 | 0.1 | 0.2 | 0.3 |
| ethane | 0.2 | 0.2 | 0.3 | 0.3 |
| ethylene | 0.3 | 0.4 | 0.5 | 0.5 |
| propane | 2.5 | 3.4 | 2.3 | 1.8 |
| propylene | 6.3 | 8.6 | 9 | 8.7 |
| C4 paraffins | 1.8 | 2.3 | 1.6 | 1.2 |
| C4 olefins | 7.4 | 9.7 | 9 | 8.6 |
| C5-C9 | 2.8 | 3.1 | 3.4 | 1.4 |
| paraffins | | | | |
| C5-C9 olefins | 8.5 | 8 | 8.1 | 6.3 |
| C1-C9 | 29.9 | 35.8 | 34.4 | 29.1 |
| paraffins + | | | | |
| olefins | | | | |
| C1-C9 | 0.1 | 0 | 0.1 | 0.1 |
| aromatics | | | | |
| C10-C20 | 69 | 63 | 65 | 70 |
| Sum | 100 | 100 | 100 | 100 |

As can be seen from Table 6, the present catalytic cracking process provides a variety of different cracking product fractions. For the sake of simplicity, C10-C20 hydrocarbons are considered here as unconverted hydrocarbon feed, as the hydrocarbon feed samples contained around 100 wt-% hydrocarbons having a carbon number of at least C10. The amount of this unconverted fraction was relatively high using the selected test run conditions and catalyst. However, the conversion can be increased by recycling the fraction of C10-C20 hydrocarbons, or a portion of it, back to the catalytic cracking. The unconverted fraction may have an increased isoparaffin content compared to that of the fresh hydrocarbon feed sample. As the propylene yield increases along the increasing isoparaffin content of the catalytic cracking feedstock, recycling the unconverted fraction may further increase the propylene yields. Additionally, an unconverted fraction with increased isoparaffin content (compared to the corresponding fresh feed) is usable as a component for aviation and/or diesel fuel compositions. Said increased isoparaffin content will contribute to good cold properties of said aviation and/or diesel fuel compositions.

TABLE 7
Conversion normalised yields of catalytic cracking
products for hydrocarbon feed samples P1-P4

| | (mol-%) | | | |
|---|---|---|---|---|
| | P1 | P2 | P3 | P4 |
| | (8 wt-% | (69 wt-% | (92.5 wt-% | (95.25 wt-% |
| | iP) | iP) | iP) | iP) |
| H₂ | 2.6 | 2.5 | 3.6 | 5.5 |
| methane | 0.3 | 0.3 | 0.6 | 1.0 |
| ethane | 0.6 | 0.5 | 0.8 | 1.0 |
| ethylene | 1.0 | 1.1 | 1.4 | 1.6 |
| propane | 8.1 | 9.3 | 6.4 | 5.8 |
| propylene | 20.5 | 23.4 | 25.1 | 28.2 |
| C4 paraffins | 5.8 | 6.3 | 4.5 | 3.9 |
| C4 olefins | 24.0 | 26.4 | 25.1 | 27.8 |
| C5-C9 paraffins | 9.1 | 8.4 | 9.5 | 4.5 |
| C5-C9 olefins | 27.6 | 21.8 | 22.6 | 20.4 |
| C1-C9 paraffins + | 97.1 | 97.5 | 96.1 | 94.2 |
| olefins | | | | |
| C1-C9 aromatics | 0.3 | 0.0 | 0.3 | 0.3 |

As can be seen from Table 7, the present catalytic cracking process provides a broad variety of different cracking product fractions with good conversion normalised yields. Especially propylene can be obtained with very high conversion normalised yields. As is evident from Table 7, the present catalytic cracking process provides also C5-C9 olefins with very high conversion normalised yields, these being usable e.g. for metathesis reactions, as comonomers in polymers, and when manufacturing lube oil additives, surfactants, agricultural chemicals, coating or corrosion inhibitors. The obtained C5-C9 paraffins, alone or combined with C5-C9 olefins, are e.g. usable as safe components, due to their low level of impurities, such as negligible aromatics content, for gasoline and/or for chemical products intended for industry or households, such as in solvents, thinners and spot removers. The present catalytic cracking process also provides C4 olefins in very high conversion normalised yields, these being usable e.g. for alkylate production, and for separating individual C4 olefins e.g. for use as (co)monomers in polymers. Furthermore, Table 7 shows that only negligible amounts of methane (a strong greenhouse gas) and aromatics (abundance of which might hinder use e.g. in solvents) are generated. The negligible formation of methane contributes to the environmental sustainability of the present catalytic cracking process.

TABLE 8
Molar ratios of selected catalytic cracking
products for hydrocarbon feed samples P1-P4

| | (molar ratios) | | | |
|---|---|---|---|---|
| | P1 | P2 | P3 | P4 |
| | (8 wt-% | (69 wt-% | (92.5 wt-% | (95.25 wt-% |
| | iP) | iP) | iP) | iP) |
| Propylene/total | 0.7 | 0.7 | 0.8 | 0.8 |
| C3 | | | | |
| Propylene/Ethene | 21.0 | 21.5 | 18.0 | 17.4 |
| C4=/total C4 | 0.8 | 0.8 | 0.8 | 0.9 |

As can be seen from Table 8, the present catalytic cracking process provides C3 fractions having high molar ratios of propylene to total C3, of at least 0.7. These C3 fractions are high quality propylene compositions of refinery grade purity. Propylene compositions of chemical grade purity, such as containing about 90-95 wt-% propylene, or of polymer grade purity containing about 99 wt-% or more propylene, are obtainable from these high quality refinery grade C3 fractions through additional purification steps. Due to the high molar ratios of propylene to total C3, additional purification steps of these C3 fractions to obtain chemical or polymer grade propylene compositions require less expensive equipment and less energy (compared to fractions having lower molar ratios of propylene to total C3). As can also be seen from Table 8, the present catalytic cracking process provides C4 fractions having high molar ratios of C4 olefins to total C4, of at least 0.8. C4 olefins are obtainable from these C4 fractions with additional purification steps requiring, due to the high molar ratios of C4 olefins to total C4, less expensive equipment and less energy (compared to fractions having lower molar ratios of C4 olefins to total C4). The very high propylene to ethylene ratios indicate that only small amounts of the hydrocarbon feed is lost in formation of less valuable (compared to propylene and/or C4 olefins) ethylene when using the present process.

TABLE 9
Conversion normalised yields of selected catalytic cracking
product fractions for hydrocarbon feed samples P1-P4

| | (mol-%) | | | |
|---|---|---|---|---|
| | P1 | P2 | P3 | P4 |
| | (8 wt-% | (69 wt-% | (92.5 wt-% | (95.25 wt-% |
| | iP) | iP) | iP) | iP) |
| total propylene | 44.5 | 49.8 | 50.2 | 56.0 |
| and C4 olefins | | | | |
| total C3-C4 | 58.4 | 65.4 | 61.1 | 65.7 |
| total C4 | 29.8 | 32.7 | 29.6 | 31.7 |
| total C2 | 1.6 | 1.6 | 2.2 | 2.6 |
| total C5-C9 | 36.7 | 30.2 | 32.1 | 24.9 |

As can be seen from Table 9, the present catalytic cracking process provides C3-C4 (total) fractions and propylene-C4 olefin fractions with very high conversion normalised yields. Fractions comprising C4 olefins, or both propylene and C4 olefins, may be used as such or after purification e.g. for producing alkylate, a high octane gasoline component, by reacting with isobutene. As can also be seen from Table 9, the present catalytic cracking process provides C5-C9 (total) fractions with very high conversion normalised yields, these fractions being usable as safe components, due e.g. to their negligible aromatics content, for gasoline and/or for chemical products intended for industry or households, such as in solvents, thinners and spot removers. The very low conversion normalised yields of total C2 indicate that a very small amount of the hydrocarbon feed is lost in formation of less valuable (compared to propylene and/or C4 olefins) ethane and ethylene, when using the present process.

### Fixed Bed Catalytic Cracking—Recycled Catalytic Cracking Feedstock

Same fixed bed catalytic cracking setup as described under Fixed Bed Catalytic Cracking of Hydrocarbon Feed was used. Again, the catalyst material was ZSM-5 prepared as described under Fixed Bed Catalytic Cracking of Hydrocarbon Feed.

P1 was converted once through under following conditions: reaction temperature 400° C., pressure 0.1 MPa, and WHSV (mass flow of the catalytic cracking feedstock/catalyst mass) 0.6 g catalytic cracking feedstock/g catalyst per hour. The liquid cracking product was collected and used as the feed in the next run (conditions: T=400° C., P 0.1 MPa, and WHSV 0.6 g catalytic cracking feedstock/g catalyst per hour).

The propylene yield was 2.1 wt-% in the once through reaction product (at the given time on stream), and the propylene yield was 9.4 wt-% in the product of the next run. This shows that the share of propylene in the cracking product can be increased by recycling at least a portion of the cracking product to the catalytic cracking feedstock.

### Fixed Bed Catalytic Cracking of Hydrocarbon Feed Using SAPO-34

Same fixed bed catalytic cracking setup as described under Fixed Bed Catalytic Cracking of Hydrocarbon Feed was used. The catalyst was prepared as described under Fixed Bed Catalytic Cracking of Hydrocarbon Feed, but this time using SAPO-34 as the catalyst material. 30 g of the catalyst was used in each experiment. SAPO-34 is a silicoalumina phosphate microporous material, a tridimensional 8-member ring molecular sieve with pore sizes of about 4 Å and having medium strong acidity.

P1 and P2 were converted once through under following conditions: reaction temperature 400° C., pressure 0.1 MPa, and feed (g/h) as indicated in Table 9-1. In an additional test P1 was converted once through under following conditions: reaction temperature 400° C., pressure 0.1 MPa, and WHSV 0.6 g catalytic cracking feedstock/g catalyst per hour, and thereafter the liquid cracking product was collected and used as catalytic cracking feedstock in subsequent run at T=400° C., p 0.1 MPa, and WHSV 0.6 g catalytic cracking feedstock/g catalyst per hour. The results are presented in table 9-1.

TABLE 9-1
Molar ratios of selected catalytic cracking products
for hydrocarbon feed samples P1 and P2 (once-through),
and for P1 liquid cracking products as feed

| | P1 | P2 | Recycle |
|---|---|---|---|
| | (8 wt-% iP) | (69 wt-% iP) | of P1 |
| Feed (g/h) | 17.5 | 17.5 | 16.7 |
| Ethylene/total C2 molar ratio | 0.9 | 0.9 | 1.0 |
| Propylene/total C3 molar ratio | 0.8 | 0.8 | 0.8 |
| Propylene/Ethene molar ratio | 7.1 | 8.7 | 9.5 |
| C4=/total C4 molar ratio | 0.7 | 0.7 | 0.7 |

As can be seen from Table 9-1, the present catalytic cracking process using SAPO-34 provides C3 fractions having high molar ratios of propylene to total C3, of at least 0.8, and C4 fractions having high molar ratios of C4 olefins to total C4, of at least 0.7. With SAPO-34 somewhat lower propylene to ethylene molar ratios are obtainable compared to ZSM-5, but strongly favouring propylene formation over ethylene formation.

Additionally thermal tests of SAPO-34 were conducted to show the stability of SAPO-34 at elevated temperatures. The thermal stability test of SAPO-34 was made by treating the catalyst at temperatures of 500° C. and 700° C. in air for 12 hours. No essential changes in the pore volume or BET surface area were observed.

### Comparison Data

As comparison data, results from Table 2 of WO2009130392A1 are provided in Table 10 below. The experiments were conducted using fixed bed catalytic cracking under conditions indicated in Table 10. Most of the catalysts used in the experiments of Table 10 contained zeolite. Table 10 reproduces results of experiments 1-4, 6, 7 and 9 of WO2009130392A1 using as the catalytic cracking feedstock non-isomerised HDO treated animal fat (Exp. 1, 2, 7, and 9) or hydrogenated palm oil (Exp. 3, and 4), or plain n-C16 (Exp. 6). Experimental and other details relating to the experiments of Table 10 are provided in WO2009130392A1, pages 8-18. Table 10. Results from Table 2 of WO2009130392A1.

TABLE 10
Results from Table 2 of WO2009130392A1.

| | Exp. 1 | Ex 2 | | | | Exp. 7 | Exp. 9 |
|---|---|---|---|---|---|---|---|
| | HDO | HDO | Exp. 3 | Exp. 4 | | HDO | HDO |
| | treated | treated | hydrogenated | hydrogenated | Exp. 6 | treated | treated |
| | animal fat | animal fat | palm oil | palm oil | n-C16 | animal fat | animal fat |
| T (° C.) | 350 | 350 | 350 | 350 | 350 | 350 | 350 |
| p (bar(a)) | 4 | 4 | 10 | 10 | 10 | 4 | 4 |
| Feed flow (g/h) | 3.0 | 3.0 | 6.8 | 6.0 | 11.0 | 3.0 | 3.0 |
| Conversion < 221 | 96.30 | 93.30 | 88.20 | 86.80 | 84.10 | 73.10 | 7.80 |
| Methane (wt-%) | 0 | 0.1 | 0 | 0 | 0 | 0 | 0 |
| Ethane (wt-%) | 0 | 0.1 | 0 | 0 | 0 | 0 | 0 |
| Ethene (wt-%) | 0.30 | 0.60 | 0.10 | 0.10 | 0.20 | 0.40 | 0.00 |
| Propane (wt-%) | 2.60 | 5.20 | 2.00 | 1.90 | 4.20 | 5.50 | 0.20 |
| Propylene (wt-%) | 4.40 | 4.10 | 1.80 | 1.60 | 1.70 | 4.70 | 0.30 |
| Butane (wt-%) | 5.80 | 8.40 | 4.60 | 4.40 | 5.80 | 5.90 | 0.20 |
| i-Butane (wt-%) | 16.70 | 23.00 | 11.00 | 10.10 | 3.50 | 3.70 | 0.70 |
| iC4 = (wt-%) | 5.80 | 4.00 | 3.20 | 2.90 | 2.80 | 4.90 | 0.30 |
| 1C4 = (wt-%) | 1.40 | 1.00 | 1.90 | 1.50 | 1.90 | 0.00 | 0.00 |
| cis-2C4 = (wt-%) | 2.10 | 1.60 | 1.10 | 1.00 | 0.90 | 1.70 | 0.10 |
| trans-2C4 = (wt-%) | 3.00 | 2.30 | 0.40 | 0.50 | 0.20 | 3.70 | 0.20 |
| butadiene (wt-%) | 0.00 | 0.00 | 0.00 | 0.00 | 0.00 | 0.00 | 0.00 |
| C4 = total (wt-%) | 12.30 | 8.90 | 6.60 | 5.90 | 5.80 | 10.30 | 0.60 |
| Propylene/total C3 | 0.63 | 0.44 | 0.47 | 0.46 | 0.29 | 0.46 | 0.60 |
| C4=/total C4 | 0.35 | 0.22 | 0.30 | 0.29 | 0.38 | 0.52 | 0.40 |
| Aromatics (wt-%) | 3.6 | 1.6 | 3.4 | 6.3 | 9.7 | 2.2 | 0.4 |

As can be seen from the comparison data in Table 10, the yields of propylene and C4 olefins are far lower compared to those obtainable by the examples according to the present disclosure. Also the ratios of propylene to total C3, and ratios of C4 olefins to total C4 are far lower compared to those obtainable by the examples according to the present disclosure. Consequently, e.g. propylene compositions of even refinery grade purity may not be achieved without additional efforts to reduce the amount of propane in the C3 fraction, and purification treatments required for obtaining any desired higher purity grade propane composition will require more expensive equipment and higher energy consumption (compared to C3 fractions obtained with the method of the present disclosure). Additionally, high amounts of aromatics are formed, limiting use of aromatics containing cracking product fractions. For example, cracking product fractions with a high aromatics content are undesired for recycling back to the catalytic cracking feedstock as aromatics increase coke formation in the catalytic cracking reaction and as aromatics are not converted into propylene. Cracking product fractions with a high aromatics content are also undesired in chemical products intended for industry or households, such as in solvents, thinners and spot removers, where safety to humans is important, as aromatics have been linked to health issues.

Various embodiments have been presented. It should be appreciated that in this document, words comprise, include and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1-24. (canceled)

25. A method for producing a cracking product fraction including propylene, C4 olefins, or both, the method comprising:
providing a catalytic cracking feedstock including:
a hydrocarbon feed including, based on a total weight of the hydrocarbon feed, at least 5 wt-% isoparaffins, a sum of a wt-% amounts of isoparaffins and n-paraffins in the hydrocarbon feed being at least 80 wt-%;
subjecting the catalytic cracking feedstock to catalytic cracking in a catalytic cracking reactor at a temperature selected from a range from 300° C. to 450° C. in a presence of a solid catalyst to obtain a cracking product; and
separating from the cracking product at least a fraction including propylene, C4 olefins, or both.

26. The method according to claim 25, comprising:
performing the catalytic cracking without feeding added molecular hydrogen (H₂) to the catalytic cracking reactor; and/or
performing the catalytic cracking without feeding added steam or added water (H₂O) to the catalytic cracking reactor.

27. The method according to claim 25, wherein the hydrocarbon feed comprises:
isoparaffins at least 8 wt-% and/or at least 10 wt-% based on a total weight of the hydrocarbon feed.

28. The method according to claim 25, wherein the hydrocarbon feed comprises:
based on a total weight of the hydrocarbon feed, at least 50 wt-% hydrocarbons having a carbon number of at least C10.

29. The method according to claim 25, wherein the hydrocarbon feed comprises:
based on a total weight of the hydrocarbon feed, at most 5 wt-% hydrocarbons having a carbon number of at least C22.

30. The method according to claim 25, wherein a sum of wt-% amounts of isoparaffins and n-paraffins in the hydrocarbon feed is at least 85 wt-% based on a total weight of the hydrocarbon feed.

31. The method according to claim 25, wherein the hydrocarbon feed is a renewable hydrocarbon feed having a biogenic carbon content of at least 90 wt-% based on a total weight of carbon in the hydrocarbon feed.

32. The method according to claim 25, comprising:
performing the catalytic cracking at a pressure selected from a range from 0.01 MPa to 5.0 MPa.

33. The method according to claim 25, comprising:
performing the catalytic cracking at a temperature selected from a range from 350° C. to 450° C.; and/or
at a temperature selected from a range from 370° C. to 450° C., or at a temperature selected from a range from 350° C. to 430° C.

34. The method according to claim 25, comprising:
selecting a weight hourly space velocity (WHSV, mass flow of the catalytic cracking feedstock/catalyst mass) of the catalytic cracking from a range from 0.01 g to 10 g catalytic cracking feedstock/g catalyst per hour.

35. The method according to claim 25, comprising:
controlling one or more of temperature, pressure and WHSV so that a conversion of catalytic cracking feedstock in the catalytic cracking is within a range from 0.20 to 0.85.

36. The method according to claim 25, wherein the solid catalyst comprises:
zeolite or zeolite type material having a micropore size within a range from 4 to 6 Å.

37. The method according to claim 36, wherein the zeolite or zeolite type material comprises:
ZSM-5, MCM-22, SAPO-34 and/or p zeolite.

38. The method according to claim 25, wherein the hydrocarbon feed is obtained by a process comprising:
hydrotreating renewable oxygen containing hydrocarbons to produce a hydrotreatment product; and
removing vapor phase from the hydrotreatment product to obtain a vapour depleted hydrotreatment product.

39. The method according to claim 25, wherein the catalytic cracking feedstock comprises:
a recycled fraction separated from the cracking product.

40. The method according to claim 25, comprising:
separating from the cracking product a fraction of hydrocarbons having a carbon number of at least C5.

41. The method according to claim 40, comprising:
recycling at least a portion of the fraction of hydrocarbons having a carbon number of at least C5 to the catalytic cracking feedstock.

42. The method according to claim 39, wherein a sum of wt-% amounts of the hydrocarbon feed and the recycled fraction in the catalytic cracking feedstock is at least 90 wt-% based on a total weight of the catalytic cracking feedstock.

43. The method according to claim 25, comprising:
purifying the fraction including propylene, or the fraction including both propylene and C4 olefins, to obtain purified propylene composition.

44. The method according to claim 25, comprising fractionating the fraction including C4 olefins, and/or both propylene and C4 olefins, to obtain one or more of 1-butene, trans-2-butene, cis-2-butene, butadiene, and/or isobutene as fractionated composition(s).
